(19) United States
(12) Patent Application Publication
(10) Pub. No.: US 2009/0311246 A1
(43) Pub. Date: Dec. 17, 2009

(54) Method of Enhancing Remyelination in Demyelinating Diseases of the Central Nervous System

(76) Inventor: Foroozan Mokhtarian, Westhampton, NY (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 12/429,670

(22) Filed: Apr. 24, 2009

Related U.S. Application Data
(60) Provisional application No. 61/125,451, filed on Apr. 25, 2008.

(51) Int. Cl. A61K 39/395; A61K 38/16; A61K 38/06; A61K 38/08; A61K 38/10; C07K 16/00

(52) U.S. Cl. 424/130.1; 514/12; 514/18; 514/14; 514/13; 530/388.1; 530/387.3

(57) ABSTRACT

The present invention provides methods for enhancing remyelination or decreasing or inhibiting demyelination in central nervous system (CNS) of a mammalian subject by administering to the subject an effective amount of a Semliki Forest Virus (SFV) epitope E2 137-151 peptide together with a pharmaceutically acceptable carrier, or an antibody to E2 137-151 peptide (“E2 137-151 antibody”). The present invention also provides a method for treating a CNS disease, particularly, multiple sclerosis (MS), in a mammalian subject by administering to the subject a therapeutically effective amount of an E2 137-151 peptide together with a pharmaceutically acceptable carrier, or an anti E2 137-151 antibody. A polyclonal and a monoclonal E2 137-151 antibody are also provided by the present invention.

### CROSS REFERENCE TO RELATED APPLICATIONS

The present application claims benefit of U.S. Provisional Application No. 61/125,451 filed on Apr. 25, 2008.

### FIELD OF THE INVENTION

The field of invention relates to the use of an antigen-specific antibody to elicit remyelination and treatment of central nervous system (CNS) diseases.

### BACKGROUND OF THE INVENTION

The clinical signs and symptoms in multiple sclerosis (MS), one of the major demyelinating diseases of the CNS, appear to be due to impaired or loss of conduction of electrical signals as a result of myelin damage (1). Myelin is a very complex substance that wraps and surrounds the nerve fibers and it is essential for the nerves to transmit electrical signals. The primary mechanism of injury is inflammation associated with myelin damage.

### Multiple Sclerosis and The Semliki Forest Virus (SF1) Model

Pathologically MS is characterized by inflammatory demyelination, a feature similar to viral infections of the CNS. Viral infections have been implicated in the pathogenesis of this disease, and several animal models have been described (1). In this invention, we have used the nonlethal strain, A7(74), of SFV (2) as a model for MS, to study the mechanisms of demyelination and remyelination. After SFV-infection, the virus is cleared a week later, by CD8⁺ T-cell (3) and antibody responses (4). A transient demyelination occurs on days 15-21, immediately followed by complete remyelination, by day 35 post infection (5). We have shown that demyelination, and subsequent remyelination, during SFV-infection, may also be both dependent on antibody responses to viral epitopes (4,5). We have found that SFV-infection acts as a triggering agent in the induction of autoimmune reslonses to myelin protein (6).

The major surface protein of SFV that evokes the antibody responses to this virus is E2 protein (7). Studies of linear epitopes of SFV E2, have identified two major ones; with one peptide being part of the envelope and the T-helper cell epitope (E2-Tₕ) of SFV (8). Antibody responses of mice to E2 Tₕ are much higher tan to other E2 peptides, and comparable to whole SFV (4). While some antibodies to a viral epitope, which mimics a peptide of myelin oligodendrocyte glycoprotein (MOG) appear to correlate with demyelination (9), antibodies to the T-helper cell epitope, E2 Tₕ was found to correlate with remyelination (current patent findings). Fast, effective and complete remyelination is a unique feature of the SFV model and, using KO mice we have found that antibody to E2 Tₕ peptide is involved in remyelination after SFV-infection.

### Multiple Sclerosis (MS) and its Autoimmune Models

MS is an inflammatory demyelinating disease of the CNS, which has been extensively studied and many therapeutic strategies have been implemented (10, 11). MS is at least partially caused by an autoimmune attack on three major proteins of myelin: myelin basic protein (MBP), proteo-lipid protein (PLP)(12-14) and myelin oligodendrocyte glycoprotein (MOG) (15-18). An important model for MS is experimental autoimmune encephalomyelitis (EAE), in which autoreactive T-cells specific for above antigens enter the CNS, cause inflammation and recruit macrophages, resulting in the destruction of myelin (10-26). Encephalitogenic epitopes of these proteins have been used to induce experimental autoimmune encephalomyelitis (EAE) in animals. MOG-induced EAE, as in the MBP- and PLP-induced models in mice, was found to be a demyelinating encephalomyelitis resembling MS. Adoptive transfer of T-cells, specific for MBP (20-23) provided useful models to study MS, and to test the therapeutic activity of potential treatment protocols (24, 25). Antibodies to MBP have been found to exert a suppressive effect on EAE induction (26).

### TCR γ/δ⁺ T Cells

TCRγδ⁺ T cells comprise only 0.5-10% of the TCR population in human peripheral blood (27), while 3% of T cells in murine spleen and lymph nodes and 0.4-4% of T cells in the brains of normal mice are TCRγδ⁺ (28). Recent studies have suggested a strong role for human peripheral blood γδ⁺ T cells in humoral immunity and to provide B cell help for antibody production (29). However, the clonotypes of B cells were altered and the majority of antibodies produced used K light chain instead of λ₁, which dominates in IgH^{b} mice (30). In B6 mice, in the absence of αβ T cells: γδ⁺ T cells were able to induce demyelination and antibody response to a T-dependent antigen (31) γδ⁺ T cells have been shown to be MHC-independent and mediate cellular immune functions without the need for antigen processing by APC (31). Some γ/δ T cells, however, have been shown to respond to non-protein antigens (32-34), such as mycobacterial lipids and glycolipids (35). In many cases, γδ⁺ cells show a broad cross-reactivity that is not seen for αβ alloreactive T cells, suggesting a fundamental difference in antigen recognition between αβ and γδ T-cells (36). Studies have suggested that γδ T-cells regulate the immune response under a variety of immune stimuli, including autoimmunity (37).

### TCR γ/δ⁺ T Cells in MS and EAE

Demyelination in multiple sclerosis (MS) is accompanied by T lymphocyte (CD4^{+,} CD8^{+,} CD4⁻ CD8⁺, αβ and γδ) infiltration of the CNS, but the underlying mechanisms are poorly understood. γδ T cells were also found in MS lesions and were thought to respond to heat shock proteins (38). While some older studies in mice have found a potentiating effect for γδ T-cells on the severity of EAE (39), others have suggested a suppressive effect (45). Depletion of γδ T-cells, by monoclonal antibody (Ab), UC7-13D5, exacerbated the recurrence of EAE induced by guinea pig spinal cord homogenate in B10.PL mice (40). We (unpublished studies) and others (39-41) have found that TCR γδ⁺ T cells are only a minor population of infiltrating cells in the CNS, and that their proportion does not significantly change during the course of CNS disease. In summary, γδT-cells may not play a role in the initiation of EAE but regulate inflammation in the CNS and promote disease recovery (42), as evidenced by a delay in the effecter-phase mechanisms, in γδ-depleted mice (39-43).

### TCRγδ T Cells in Viral Infections

It has also been found that γδ⁺T-cells are involved in the immune response against viruses such as herpes simplex (44), vaccinia (45), Coxsackie B (46), and vesicular stomatitis virus (47). Mouse hepatitis virus (MHV)-induced demyelination, in nude mice, was mediated by γδ⁺ T-cells, which substituted for the usual αβ⁺ T-cells, in this process (30). In influenza virus infection of mice the TCR γδ response occurred after the initial TCR αβ response (48) and these cells accumulated in inflammatory lesions in the late stages of infection, after the clearance of the virus. The above studies in EAE and some viral infections, though not quite similar, share the finding that TCRγδ T cells may play a role in the recovery and repair of the CNS damage following inflammation and prompted us to study the role of these cells in remyelination following SFV-infection.

### SFV-Model of MS and Role of Molecular Mimicry

Molecular mimicry, or antigenic cross reactivity, between proteins of the virus and those of myelin may be the mechanism responsible for cross recognition and could lead to destruction of myelin following an antiviral immune response (49). The activation of autoantigen-reactive human T cells by viral peptides have provided evidence for a role of molecular mimicry between viral and autoantigenic peptides in the pathogenesis of human demyelinating and autoimmune and diseases (50-52), such as multiple sclerosis (MS) (50), diabetes (53) and systemic lupus erythematosus (54). A frequent sequela of viral infections of the central nervous system (CNS) is the generation of viral specific antibodies that cross-react with constitutive epitopes found within the CNS (55).

We have previously shown that infection of B6 mice with SFV triggered susceptibility to the induction of EAE, and this effect was transferred to naive mice (6). We have also found amino acid (aa) homologies (mimicry) between the SFV (E2) and proteins of myelin (MOG, and MBP) (9). E2 is the major surface glycoprotein of SFV containing the T cell epitopes for immune responses (8). This mimicry consisted of some areas of 3 consecutive complete homologies (combined with some partial) in the regions of T-cell epitopes of E2. Inoculation of mice with MBP mimicked peptide of E2 did not cause histopathology in mice (unpublished data), but inoculation with mimicked peptide of MOG did (9).

Significance and Relevance of SFV model to Treatment of MS

There are several unique aspects of SFV model; one can easily initiate infection with SFV in the periphery and induce CNS disease, and demyelination occurs after viral clearance. These features are in contrast to the other two viruses; Theiler's and MHV that establish persistent and chronic CNS infections. Given the complex and unknown etiology of MS, it would be wise to study both type of models. The most important feature of the SFV model is that it quickly, efficiently and fully remyelinates after the autoimmune mediated transient demyelination (5).

### Role of Antibody in Remyelination

Theiler's murine encephalomyelitis virus (TMEV) infection of mice is a persistent demyelinating virus of the CNS (51), which is widely used to study demyelination and remyelination. Previous experiments with TMEV showed that only 4 to 5% of the demyelinated area exhibited significant spontaneous remyelination (56). In protocols using therapy with mouse monoclonal IgM antibody against spinal cord homogenates, this number increased four-fold (57). Using the TMEV, EAE and Lysolecithin-induced demyelinating (58, 59) models, it has been demonstrated that the passive transfer of CNS specific antiserum and purified monoclonal antibodies directed against myelin components promoted CNS remyelination (57, 58). Researchers also isolated a monoclonal IgM antibody from human serum that reacted against a surface component of oligodendrocytes and promoted remyelination (60). It has also been shown that antibodies reactive with MBP promoted CNS remyelination (61). MBP domains are thought to be involved in myelin compaction, and of cytoskeleton of myelin membrane lamellae (62), Treatment with these mouse and human antibodies (63) suggested that remyelinating-promoting antibodies might bind to the surface of oligodendrocytes or astrocytes thereby inducing Ca⁺⁺ signals and subsequent physiologic effects. Another study demonstrated that the remyelination-promoting activity of antibody was not dependent on immunomodulation (64). During SFV-infection, antibodies may mediate remyelination by binding to a unique receptor on CNS cells. In this respect, antibodies can exert their influence by blocking or stimulating function (65). More recent studies suggested that antibodies directed against myelin induced antiapoptotic signaling in premyelinating oligodendrocytes in mice undergoing antibody-induced remyelination (66). Cross-reactivity between antibodies to MBP and to copolymer 1, which has suppressive effects on EAE, has been established. This finding has suggested a role for anti MBP antibodies in the suppression of MS (67, 68).

This provisional patent study has shown that viral-induced antibody response to a peptide of E2, which has mimicry with a peptide of MBP, is involved in remyelination. Immunization with this peptide that increased the antibody to this peptide only, promoted remyelination in KO mice. Elucidation of treatment protocols by which this remyelination-promoting antibody exert its beneficial effect is worthwhile in the overall development of targeted therapies for MS, especially with regard to heterogeneity in the etiologies of demyelination and patterns of remyelination in different forms of MS (69).

### SUMMARY OF THE INVENTION

The present invention identifies a viral epitope of Semliki Forest Virus (SFV), E2 137-151 (hereinafter “E2 137-151 peptide” or “E2 137”), which mimics a peptide of mouse and human myelin basic protein (MBP). The present invention recognizes that antibodies to E2 137-151 peptide are involved in establishing and/or enhancing the remyelination of the damaged myelin sheath that coats axons in the CNS. The present invention also recognizes that antibodies to E2 137-151 peptide are involved in reducing the demyelination of myelin sheath that coats axons of the CNS.

Accordingly, one aspect of the present invention is directed to a method of inducing, establishing and/or enhancing remyelination in the CNS of a mammalian subject by administering to the subject an effective amount of SFV epitope, preferably E2 137-151 peptide, or homolog thereof, and a pharmaceutically acceptable carrier.

Another aspect of the present invention is directed to a method of inducing, establishing and/or enhancing remyelination in the CNS of a mammalian subject by administering to the subject an effective amount of an anti-E2 137-151 peptide antibody (“E2 137-151 antibody”) or an antibody against a homolog of E2 137-151 peptide (“E2 137-151 homolog antibody”).

In a particular aspect, the present invention contemplates a method of inducing, establishing and/or enhancing remyelination in the CNS of a mammalian subject by administering to the subject an effective amount of E2 137-151 peptide or homolog thereof together with a pharmaceutically acceptable carrier, and an E2 137-151 antibody or E2 137-151 homolog antibody.

In still another aspect, the present invention is directed to a method for reducing, decreasing, ameliorating and/or inhibiting demyelination in the CNS of mammalian subject by administering an effective amount of E2 137-151 peptide or homolog thereof, and a pharmaceutically acceptable carrier. Alternatively, the present invention is directed to a method of reducing, decreasing, ameliorating and/or inhibiting demyelination in the CNS of a mammalian subject by administering to the subject an effective amount of an E2 137-151 antibody or E2 137-151 homolog antibody. The present invention also contemplates a method of reducing, decreasing, ameliorating and/or inhibiting demyelination in CNS of a mammalian subject by administering to the subject an effective amount of E2 137-151 peptide or homolog thereof together with a pharmaceutically acceptable carrier, and an E2 137-151 antibody or E2 137-151 homolog antibody.

In yet another aspect, the present invention is directed to a method of treating a CNS disease manifesting the clinical characteristics associated with damaged myelin, demyelination of myelin, or destruction of myelin sheath coating axons of the CNS in a mammalian subject by administering to the subject a therapeutically effective amount of E2 137-151 peptide or homolog thereof, and a pharmaceutically acceptable carrier. Alternatively, the present invention is directed to a method of treating a CNS disease manifesting the clinical characteristics associated with damaged myelin, demyelination of myelin, or destruction of myelin sheath coating axons of the CNS in a mammalian subject by administering to the subject a therapeutically effective amount of an E2 137-151 antibody or E2 137-151 homolog antibody. The present invention also contemplates a method of treating a CNS disease manifesting the clinical characteristics associated with damaged myelin, demyelination of myelin, or destruction of myelin sheath coating axons of the CNS in a mammalian subject by administering to the subject a therapeutically effective amount of E2 137-151 peptide or homolog thereof together with a pharmaceutically acceptable carrier, and an E2 137-151 antibody or E2 137-151 homolog antibody.

In still yet another aspect, the administration of the E2 137-151 peptide and/or homolog thereof in accordance with the present invention further induces, increases, or enhances the production of γδ T-cell receptor (TCR γδ) T cells in the subject simultaneously with, or sequentially to, the administration of the E2 137-151 peptide and/or homolog thereof.

An E2 137-151 peptide or homolog thereof employed in accordance with the present invention can be in the form of a single E2 137-151 peptide or homolog molecule as well as in the form of polymer of E2 137-151 peptide or polymer of E2 137-151 peptide homolog molecules.

The homolog of E137-151 peptide contemplated by the present invention can be any peptide molecule that mimics a mammalian myelin protein, including, but not limited to, mouse MBP 56-68 peptide and human MBP 102-118 peptide.

A particular CNS disease contemplated by the methods of the present invention is multiple sclerosis (MS).

In a particular aspect of the present invention, the E2 137-151 peptide or homolog thereof is administered subcutaneously.

In another particular aspect of the present invention, the E2 137-151 antibody or E2 137-151 homolog antibody contemplated by the present invention is administered intravenously or intraperitoneally.

In a further aspect, the E2 137-151 antibody or E2 137-151 homolog antibody contemplated by the present invention is polyclonal. In another further aspect, the E2 137-151 antibody or E2 137-151 homolog antibody contemplated by the present invention is monoclonal. In another further aspect, the E2 137-151 antibody or E2 137-151 homolog antibody contemplated by the present invention is humanized monoclonal.

### BRIEF DESCRIPTION OF THE DRAWINGS

FIG. 1 Comparison of TCR γδ expression on CD3⁺CD4SR⁻ mononuclear cells isolated from brain and spleen tissue during SFV infection of wild-type (A. WT) and γδ-knockout (B. γδ-KO) mice.

Mice were inoculated intraperitoneally with 10⁴ PFU of SFV (A7). Mononuclear cells were isolated, stained with specific and isotype-matched control antibodies, as described infra, and analyzed by three-color flow cytometry. 10⁴ events were collected and mononuclear cells were selected according to FSC/SSC, differentially sorted by the expression of CD3 and CD45R, and the percentage of T-cells (CD3⁺CD45R⁻) that expressed TCR γδ determined. The data shown are from one representative experiment of three, with each observation consisting of pooled cells from three to five mice. Only BMNC stained with the specific (anti-TCR γδ; GL3) antibody are shown. Values of isotype-matched antibody for TCR γδ, were 1% for do, and <1% for other days (not shown in this figure).

FIG. 2 Inflammation, demyelination and remyelination in SFV-infected WT and γδ-KO B6 mice.

All figures come from one micron epoxy sections taken from brain tissue fixed in 2.5% glutaraldehyde/1% osmium tetroxide, embedded in epoxy resin, stained with toluidine blue and photographed by light microscopy.

A. Brain Stem; SFV-infected WT, 21 days post-infection (pi). Perivascular cuffs of lymphocytes and macrophages are seen around blood vessels (v). Collections of microglial cells and macrophages are also seen (arrows). X150.

B. Brain Stem; SFV-infected VIT day 21 pi. Dernyelination is present (arrows). Some reactive microglia are seen (m). X180.

C. Cerebellum; SFV-infected WT; day 35 pi. The white matter layer of cerebellum displays complete remyelination (dark myelin sheaths). X120.

D. Brain stem; SFV-infected γδ-KO mice, day 35 pi. In this area of white matter, residual inflammatory activity (perivascular cuff at v), demyelination (arrows) and some early remyelination (thin myelin sheaths, light color) are shown. X150.

E. Corpus callosum; SFV-infected WT day 42 pi. The white matter layer displays widespread remyelination. X180

F. Corpus callosum, SFV-infected γδ-KO mice, day 42 pi. Demyelinated fibers are still present (arrows). Some of the longitudinally-oriented callosal fibers display very thin myelin sheaths, reminiscent of very early remyelination. X 180.

FIG. 3 Antibody responses in the sera (A&B) of SFV-infected wild-type (WT) and γδ-knockout (KG) mice, immunized with E2 137-151. Sera were harvested at various times after infection and reacted to; SFV and to E2 peptide, 137-151 (A&B), and PPD (data not shown).

Optical densities (OD) (3A) and ratios (3B) of Non-immunized/Immunized, WT and KO, of ELISA responses to SFV and to E2 peptide, 137-151 is shown. Samples were collected on days 15, 21, 28 and 35 after intraperitoneal infection of B6 mice with SFV and immunization with E2 137-151. The dilutions for sera were 1:80, Amount of antibody is shown as mean optical density (OD) of sera from three experiments±standard error of the mean (SEM). The background OD is subtracted from each time point.

FIG. 4 Remyelination in E2 137-151 immunized, SFV-infected WT and γδ-KO B6 mice. All figures come from one micron epoxy sections taken from brain tissues, fixed in 2.5% glutaraldehyde/1% osmium tetroxide, embedded in epoxy resin, stained with toluidine blue and photographed by light microscopy.

A. Brain stem; E2 137-151 immunized, SFV-infected mice, day 28 pi. Plasma cells (arrows) are common in this tissue. X180.

B. Cerebral hemisphere; corpus callosum; E2 137-151 immunized, SFV-infected γδ-KO mice, day 28 pi. In contrast to FIG. 2 F, all of these longitudinally-oriented callosal fibers display advanced remyelination. Ventricle above, note ependymal cells. X 120.

C. Cerebral hemisphere; corpus callosum; A normally myelinated tissue is shown for comparison. Note the well-myelinated nerve fibers in cross-section. Ventricle above left, note ependyma and choroids plexus. X 120.

FIG. 5 Comparison of Clinical Scores (±SEM) of SFV-infected E2 137-151 peptide-immunized, and non-immunized TCR γδ-KO and WT mice, on different days post infection. Data shown is the average of two experiments with a total of 15 WT, 15 KO mice, and 10 immunized KO mice (only 5-10 KO mice could be obtained at each time point). SFV-infected KO mice were immunized subcutaneously with E2 137-151 (0.5 mg/injection or 25 mg/kg, based on the average weight of 20 g/mouse) in IFA, one day after SFV-infection. Additional inoculations of E2 137-151/IFA were performed on days 5, 15, and 20. Mice were sacrificed on days 7, 15, 21, and 35 pi. Clinical scores of sacrificed and dead animals have been considered in average calculation until the end of experiments on day 35. Average clinical score of WT<KO starting on day 7 pi (p≦0.05), and average clinical score of nonimmunized <E2-137 immunized KO mice starting on day 15 pi (p≦0.05). Data from E2-137-immunized WT mice is not shown (p=NS).

FIG. 6 Comparison of Average Clinical Scores (±SEM) of E2 137-151 peptide-treated, and untreated B6 mice with EAE, on different days post treatment. Data shown is the average of three experiments, total numbers of mice were 21 EAE and 22 treated EAE mice. All mice were immunized subcutaneously with myelin oligodendrocyte glycoprotein (MOG) peptide 35-55 in CFA, on D0. Treated EAE mice received subcutaneous injection of E2-137 peptide (0.5 mg/injection or 25 mg/kg, based on the average weight of 20 g/mouse) in IFA, on day 12 post EAE induction. Additional inoculations of E2-137 peptide/IFA were performed twice more with 5-day intervals. Following the injection, animals were observed daily for clinical manifestations of disease and were scored on a scale of 0-VI, as above. Clinical scores of sacrificed and dead animals have been considered in average calculation till the end of the experiments on day 39. Average disease severity in EAE mice was significantly higher than treated EAE mice on D19 (p<0.001).

FIG. 7 Comparison of antibody responses (±SEM) of EAE, E2-137-151 peptide treated, and untreated EAE mice to E2-137-151, MOG 35-55 and control peptides. Three experiments were performed with total number of 21 EAE and 22 treated EAE. All mice were immunized subcutaneously with myelin oligodendrocyte glycoprotein (MOG) peptide, 35-55 in CFA, as described above, on D0. EAE mice were treated as described for FIG. 6. All mice were sacrificed at the end of experiment on D39 post immunization.

*E2-137 peptide treated>untreated EAE mice (p<0.01).

FIG. 8 Remyelination in E2 137-151 immunized B6 mice with EAE.

All figures come from one micron epoxy sections taken from brain tissues, fixed in 2.5% glutaraldehyde/1% osmium tetroxide, embedded in epoxy resin, stained with toluidine blue and photographed by light microscopy.

A—Lumbar spinal cord of a mouse with EAE, day 33. Extensive inflammatory response is seen in the white matter. White matter also displays groups of demyelinated axons (arrows) and widespread Wallerian degeneration evidenced by dilated and collapsed myelin sheaths.

### X100.

B—A similar preparation as in FIG. 8 A. Lumbar spinal cord of E2 137-151 treated, EAE mice, day 33 pi. In contrast to untreated EAE mice, some fibers display remyelination (arrows). No wallerian degeneration and inflammation is present.

### X 100.

C—A normally myelinated tissue is shown for comparison. Note the well-myelinated nerve fibers in cross-section. X 100.

### DETAILED DESCRIPTION OF THE INVENTION

In one embodiment, the present invention is directed to a method of inducing, establishing and/or enhancing remyelination in the CNS of a mammalian subject by administering to the subject an effective amount of SFV epitope and a pharmaceutically acceptable carrier. Preferably, the SFV epitope is E2 137-151 peptide or homolog thereof. More preferably, the SFV epitope is E2 137-151 peptide having the amino acid sequence GREKFTIRPHYGKEI (SEQ ID NO: 1) or homolog thereof.

The homolog of E137-151 peptide contemplated by the present invention can be any peptide molecule that mimics a mammalian myelin protein, including, but not limited to, mouse MBP 56-68 peptide and human MBP 102-118 peptide.

By “mimic” is meant being similar to or imitate according to molecular mimicry. By “molecular mimicry” is meant the theoretical possibility that the similarity of the amino acid sequences between a peptide that is foreign to a subject and a subject's self-peptides is sufficiently enough to result in the cross-activation of the subject's body reactions by pathogen-derived peptides. For example, a homolog of E137-151 peptide results in autoreactive T cells or B cells reactions to pathogen-derived E137-151 peptide derived from SFV epitope.

In a particular embodiment, the homolog of E2 137-151 peptide in accordance with the present invention is a peptide molecule containing amino acid sequence HYG and homologs, preferably in full-length, to mouse MBP 56-68 peptide having the amino acid sequence GKDSHTRTTHYGS (SEQ ID NO: 2). In another particular embodiment, the homolog of E2 137-151 peptide in accordance with the present invention is a peptide molecule containing amino acid sequence GRE and homologs, preferably in full-length, to human MBP 102-118 peptide having the amino acid sequence GREDNTFKDRPSESDEL (SEQ ID NO: 3).

An E2 137-151 peptide or homolog thereof employed in accordance with the present invention can be in form of a single E2 137-151 peptide or E2 137-151 peptide homolog molecule as well as in form of polymer E2 137-151 peptide or polymer E2 137-151 peptide homolog molecules.

In another embodiment, the invention contemplates random copolymers of short peptides, i.e. peptides of about three to fifteen amino acids comprising HYG (HisTyrGly) with exact homology in three consecutive amino acids between SFV E2 sand MouseMBP or short peptides containing GRE (GlyArgGlu) with exact homology in three consecutive amino acids between SFV E2 and human MBP or short peptides containing GKH with exact homology in two amino acids out of three between SFV E2 and human MBP.

Glatiramer acetate (GA; Copaxone; copolymers-1) is a polypeptide compound used to treat multiple sclerosis (MS). It is a mixture of peptides of varying lengths, randomly synthesized from alanine, lysine, glutamic acid, and tyrosine at molar ratios of 6.1:4.7:1.9:1.0, respectively (63,68).

In another embodiment, the present invention is directed to a method of inducing, establishing and/or enhancing remyelination in the CNS of a mammalian subject by administering to the subject an effective amount of an anti-E2 137-151 peptide antibody (“E2 137-151 antibody”) or an antibody against a homolog of E2 137-151 peptide (“E2 137-151 homolog antibody”).

According to the present invention, a passive transfer of E2 137-151 antibody is contemplated. By “passive transfer” is meant administering an actual antibody as opposed to making or inducing it in vivo, e.g., by E2 137-151 peptide or homolog thereof. For example, a passive transfer of E2 137-151 antibody or E2 137-151 homolog antibody in mice with EAE disease can be conducted as described in Example 7. According to the present invention, immunization with peptide also works in EAE.

In a particular embodiment, the present invention contemplates a method of inducing, establishing and/or enhancing remyelination in the CNS of a mammalian subject by administering to the subject an effective amount of E2 137-151 peptide and/or homologue thereof together with a pharmaceutically acceptable carrier, and an E2 137-151 antibody and/or E2 137-151 homolog antibody.

An E2 137-151 antibody and/or E2 137-151 homolog antibody contemplated by the present invention can be polyclonal or monoclonal, preferably, monoclonal, e.g., a monoclonal antibody (mAb) to E2 137-151 peptide, a mAb to E2 137-151 peptide homologue or a humanized mAb to E2 137-151 peptide homologue.

“Remyelination” as used in connection with the present invention involves repairing damaged myelin, e.g., caused by infection or inflammation, in the CNS. Remyelination can be achieved by any means that can promote the repair of the damaged myelin, e.g., by replacing myelin producing cells or restoring their function. For example, without intending to be limited by any particular mechanism, the body's failure to repair myelin in an MS patient is believed to lead to nerve damage, which causes MS symptoms and increasing disability. It is believed that by repairing myelin, nerves can be able to send proper signals again and thereby restoring any loss of function as well as preventing further damage. Without intending to be limited by any particular mechanism, it is believed that inducing, establishing and/or enhancing remyelination can lead to the treatment of MS.

By “effective amount” is meant an amount sufficient to produce a desired effect. For example, an “effective amount” of E2-137-151 peptide or antibody can be a concentration of E2-137-151 peptide or antibody sufficient for establishing or enhancing remyelination in central nervous system (CNS) of a mammalian subject. Alternatively, an “effective amount” of E2-137-151 peptide or antibody can be a concentration of E2-137-151 peptide or antibody sufficient for decreasing, ameliorating or inhibiting demyelination in CNS of a mammalian subject.

By “therapeutically effective/efficient amount” is meant an amount sufficient to produce a desired treatment or therapy effect. For example, a therapeutically effective amount as used in connection with the present invention can be an amount of E2-137-151 peptide and/or antibody that is high enough to positively modify the condition to be treated, e.g., multiple sclerosis, but low enough to avoid serious side effects (at a reasonable benefit/risk ratio), within the scope of sound medical judgment.

According to the present invention, the precise amount or dosage of E2-137-151 peptide or E2-137-151 peptide antibody to be effective depends upon the condition of the subject that is being treated. The precise amount may depend on the weight of the subject, as well as the route of administration. As a general rule, for subcutaneous administration, regimes in cumulative amounts ranging from about 0.1 mg to about 1 mg (or about 5 mg/kg to about 50 mg/kg) exogenous E2-137-151 peptide per 5-day interval for a human patient is effective. As a general rule, for intravenous or intraperitoneal administration, regimes in cumulative amounts ranging from about 0.8 mg to about 1.2 mg of exogenous anti E2-137-151 antibody, or about 40 mg/kg to about 60 mg/kg, weekly or at 4-day intervals for a human patient is effective.

By “modulate” or “modulating” or “modulation” is meant to adjust, alter or keep a level or condition to or in a proper measure or proportion. The term “modulate” or “modulating” or “modulation” as used herein includes the inhibition or suppression of a function or activity (such as demyelination) as well as the enhancement of a function or activity (such as remyelination).

By “treat” or “therapy” is meant an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total, whether detectable or undetectable. The term “treat” or “therapy” can also mean prolonging survival as compared to expected survival if not receiving treatment.

By “inhibit,” “suppress,” “reduce,” “decrease” or “ameliorate” is meant to reduce the function or activity, such as demyelination, when compared to otherwise same conditions except for a condition or parameter of interest, or alternatively, as compared to another conditions.

As used herein, the term “cell” or “animal cell” shall be interpreted to include any cell derived from an animal, including a mammal (e.g., a human). The term encompasses cells grown in vitro, ex vivo, and those in vivo, and includes progeny of any of the above.

By “subject” is meant a mammalian organism, preferably a human.

By “CNS diseases” is meant any disease or condition in the CNS that is related to impaired or loss of conduction of electrical signals as a result of myelin damage, particularly, due to inflammation. Particularly, the CNS diseases contemplated by the present invention include inflammatory demyelinating diseases of the CNS caused by impaired or loss of conduction of electrical signals as a result of myelin damage due to inflammation. More particularly, the CNS diseases in connection with the present invention include, but are not limited to, all viral-induced demyelinating diseases of the CNS. A particular CNS disease contemplated by the methods of the present invention is multiple sclerosis (MS).

By “pharmaceutically effective/acceptable carrier” is meant a carrier that is conventionally used in the art to facilitate the storage, administration, and/or the healing effect of the agent by potentiating the immune response to the agent. A suitable carrier should be stable, i.e., incapable of reacting with other ingredients in the formulation. A suitable pharmaceutically carrier should not produce significant local or systemic adverse effect in recipients at the dosages and concentrations employed for treatment. A pharmaceutically acceptable carrier of the present invention is one that is suitable for animal, particularly, human, administration and does not include compounds that are utilized in animal toxicological studies. Such carriers are generally known in the art.

Suitable carriers for the present invention include those conventionally used, but are not limited to, albumin, gelatin, collagen, polysaccharide, monosaccharides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), and the like.

Water, saline, aqueous dextrose, and glycols are preferred liquid carriers, particularly (when isotonic) for solutions. The carrier can be selected from various oils, including, but not limited to, those of petroleum, animal, vegetable or synthetic origin, for example, peanut oil, soybean oil, mineral oil, sesame oil, and the like. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. The compositions of the present invention can be subjected to conventional pharmaceutical expedients, such as sterilization, and can contain conventional pharmaceutical additives, such as preservatives, stabilizing agents, wetting, or emulsifying agents, salts for adjusting osmotic pressure, buffers, and the like.

In still another embodiment, the present invention is directed to a method for reducing, decreasing, ameliorating and/or inhibiting demyelination in the CNS of mammalian subject by administering an effective amount of E2 137-151 peptide or homolog thereof, and a pharmaceutically acceptable carrier.

Alternatively, the present invention is directed to a method of reducing, decreasing, ameliorating and/or inhibiting demyelination in the CNS of a mammalian subject by administering to the subject an effective amount of an E2 137-151 antibody or E2 137-151 homolog antibody.

The present invention also contemplates a method of reducing, decreasing, ameliorating and/or inhibiting demyelination in CNS of a mammalian subject by administering to the subject an effective amount of E2 137-151 peptide or homolog thereof together with a pharmaceutically acceptable carrier, and an E2 137-151 antibody or E2 137-151 homolog antibody.

In yet another embodiment, the present invention is directed to a method of treating a CNS disease manifesting the clinical characteristics associated with damaged myelin, demyelination of myelin, or destruction of myelin sheath coating axons of the CNS in a mammalian subject by administering to the subject a therapeutically effective amount of E2 137-151 peptide or homolog thereof, and a pharmaceutically acceptable carrier.

Alternatively, the present invention is directed to a method of treating a CNS disease manifesting the clinical characteristics associated with damaged myelin, demyelination of myelin, or destruction of myelin sheath coating axons of the CNS in a mammalian subject by administering to the subject a therapeutically effective amount of an E2 137-151 antibody or E2 137-151 homolog antibody.

The present invention also contemplates a method of treating a CNS disease manifesting the clinical characteristics associated with damaged myelin, demyelination of myelin, or destruction of myelin sheath coating axons of the CNS in a mammalian subject by administering to the subject a therapeutically effective amount of E2 137-151 peptide or homolog thereof together with a pharmaceutically acceptable carrier, and an E2 137-151 antibody or E2 137-151 homolog antibody.

In a preferred embodiment of the present invention, the E2 137-151 peptide or homolog thereof is administered subcutaneously.

In another preferred embodiment of the present invention, the E2 137-151 antibody or E2 137-151 homolog antibody contemplated by the present invention is administered intravenously.

According to the present invention, the compositions comprising an effective amount of E2-137-151 peptide or homolog thereof and/or E2-137-151 antibody or E2-137-151 homolog antibody may be formulated into compositions having a variety of forms. The compositions of the present invention will be administered at an effective dose to induce the particular type of tissue at the treatment site selected according to the particular clinical condition addressed. Determination of a preferred pharmaceutical formulation and a pharmaceutically and therapeutically efficient/effective dose regiment for a given application is well within the skill of the art taking into consideration, for example, the administration mode, the condition and weight of the patient, the extent of desired treatment and the tolerance of the patient for the treatment. The composition can also include at least one pharmaceutically additive, carrier, or adjuvant that is suitable for administering an E2-137-151 peptide or homolog thereof.

Doses expected to be suitable starting points for optimizing treatment regiments are based on the results of in vitro, ex vivo and/or in vivo assays. Based on the results of such assays, a range of suitable E2-137-51 peptide or antibody concentrations can be selected to test at a treatment site in animal models, e.g., mice models, and then in humans.

According to the present invention, E2 137-151 antibody or E2-137-151 homolog antibody can be transferred to SFV-infected mice; E2 137-151 peptide immunization is performed in EAE mice.

The effective amount of E2-137-151 antibody that can be administered according to the present invention for an intravenous therapy is between about 0.8 to about 1.2 total. Mice will receive a total of 0.8 mg of purified anti E2 137-151 antibody, intraperitoneally or intravenously, by 8 injections of 0.1 mg each. Injections will be administered at 5 day Intervals over 40 days.

Alternatively, mice will receive a total of 1.2 mg of purified anti E2 137-151 antibody administered in 4 injections of 0.3 mg each, every 10 days, over 40 days.

Preferably, about 4.0 to about 6.0 mg/kg body weight of total antibody will be given, every 4-5 days over a period of 40 days. E2-137-151 antibody may be administered at least once a week, and as frequently as once every 5 days, throughout the entire treatment period. E2-137-151 antibody is safe and nontoxic and may be administered in essentially any amount necessary to be effective.

The effective amount of E2-137-151 antibody, E2-137-151 homologue antibody, monoclonal or humanized monoclonal anti E2 137-151 or anti E2 137-151 homolog antibody may be in a variety of forms. These include, for example, solid, semi-solid and liquid dosage forms such as tablets, pills, powders, liquid solutions or suspensions, suppositories, and injectable and infusible solutions. The preferred form depends on the intended mode of administration and therapeutic application and may be selected by one skilled in the art. Modes of administration may include systemic such as oral, parenteral (such as subcutaneous, intravenous, intraarterial, intralesional, intraosseous, intramuscular, intradermal, transdermal, transmucosal and inhalational), intraperitoneal, topical or local administration. Preferably, E2-137-151 antibody or E2-137-151 homolog antibody is administered intravenously. The compositions may be formulated in dosage forms appropriate for each route of administration.

As the skilled artisan will appreciate, lower or higher doses than those recited may be required. Specific dosage and treatment regimens for any particular patient will depend upon a variety of factors, including the activity of intravenous, the age, body weight, general health status, sex, diet, time of administration, rate of excretion, drug combination, the severity of the tissue damage, and the judgment of the treating physician.

In still yet another embodiment, the administration of the E2 137-151 peptide and/or homolog thereof in accordance with the present invention further induces, increases, or enhances the production of γδ T-cell receptor (TCR γδ) T cells in the subject simultaneously with, or sequentially to, the administration of the E2 137-151 peptide and/or homolog thereof. Without intending to be limited by any theory, it is believed that TCR γδ T cells are involved in enhancing the production of E2 137-151 antibodies and/or E2 137-151 homolog antibodies. It is also believed that TCR γδ T cells are involved in enhancing remyelination in the CNS of a mammalian subject.

The following examples are provided to describe the invention in more detail. They are intended to illustrate, not to limit the present invention in any way.

### Methods

### Infection of Mice

Female, 5 to 6-week-old C57BL/6 (WT) and B6.129P2-Tcrd^{tm1Mom} congenic mice with a disrupted TCR Cδ region (γδ KO) (Jackson Laboratory, Bar Harbor, Me.) were used in all experiments. Notably, γδ KO mice have been shown to lack TCR γδ⁺ cells in the thymus, intestine and peripheral blood of adult mice, but maintain normal numbers of TCR αβ⁺ cells. (All mice were inoculated ip with 10⁷ Tissue Culture Infective Dose 50 (TCID₅₀) units (containing 10⁴ PFU) of the avirulent SFV A7(74) 6). Mice were observed daily for clinical manifestations of disease, and scored from 0-6, as previously described (9). For all experiments animals were randomly selected and anesthetized with Metofane (Schering-Plough Animal Health, Union, N.J.) prior to sacrifice.

### Infectivity Assay: Tissue Culture Infective Dose 50 (TCID₅₀)

To measure the clearance of the SFV virus from brains of infected animals a TCID₅₀ assay was performed as we have previously described (Mokhtarian et al., 1999e); (Mahy, 1985). Briefly, brain tissue from WT and γδ KO mice, on days 0, 3, 5, 7, 10, 14 and 21 pi, were homogenized and titrated in a 96-well tissue culture plates (Costar, Cambridge, Mass.). Plates were incubated at 37° C., 5% CO₂, for 36 to 48 hours. Stock SFV A7 (74) virus was used as a positive control, and culture medium was used as a negative control. Finally the viral titers were calculated as the dilution of the virus that caused cytopathic effect (CPE) in 50% of the cultured wells and expressed as −log₁₀ TCID₅₀ units in 0.1 ml.

### Isolation of Mononuclear Cells for Flow Cytometry

A. Brain Mononuclear cells (BMNC) were purified from the brains of 3 KO and 4-6 WT, SFV-infected and noninfected mice on designated days, by digestion and Ficoll-Paque gradient centrifugation, as previously described (4).

B. Intraepithelial lymphocytes (IEL) were isolated from the small intestines of above mice, as previously described, with some modifications (70). Briefly, after transcardial perfusion, the intestine was cut at the pyloric sphincter and 1 cm proximal of the ileo-cecal junction to remove the intestine. Peyer's patches were removed and the lumen of the intestine was gently flushed with 10 ml of ice cold complete RPMI 1640. The intestine was then cut both longitudinally and into 1 cm segments. Thereafter, the pieces of intestine were incubated for 1 hour in a stirring, warm, 37° C., dissociation solution containing 1 mM DTT and 1 mM EDTA. After dissociation, the tissue was passed once over a nylon filter, and the eluate was centrifuged at 300×g for 5 min. The pellet was then resuspended in 40% Percoll, overlaid on top of a 70% Percoll gradient, and centrifuged at 600×g for 30 min. IELs were then collected from the 40%/70% interphase and washed once in PBS.

C. Spleen mononuclear cells (SMNC) were isolated from spleens of above mice by passing spleen tissue through a stainless steel mesh grid, and red blood cells were removed by an ammonium chloride lysis buffer.

All cells were quantitated by Trypan-blue exclusion and subsequently utilized for flow cytometry.

### Immunofluorescent Staining and Flow Cytometric Analysis

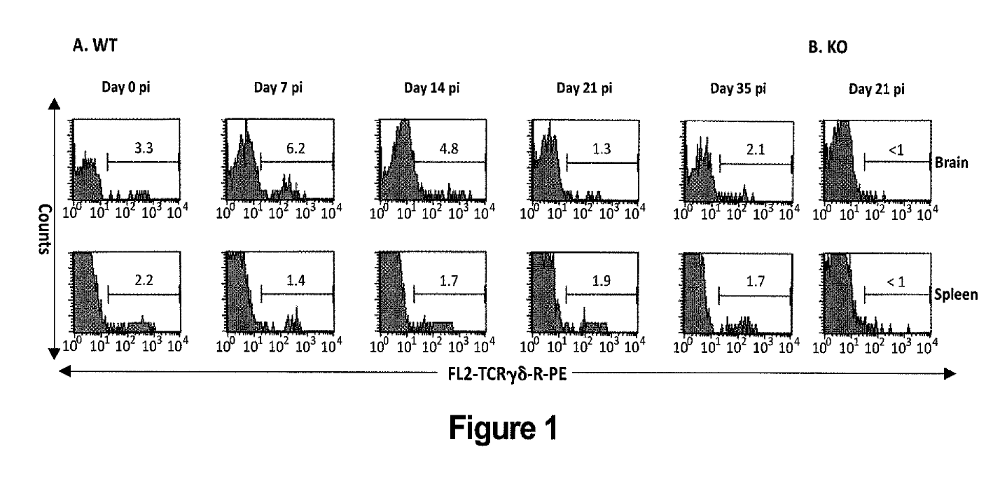

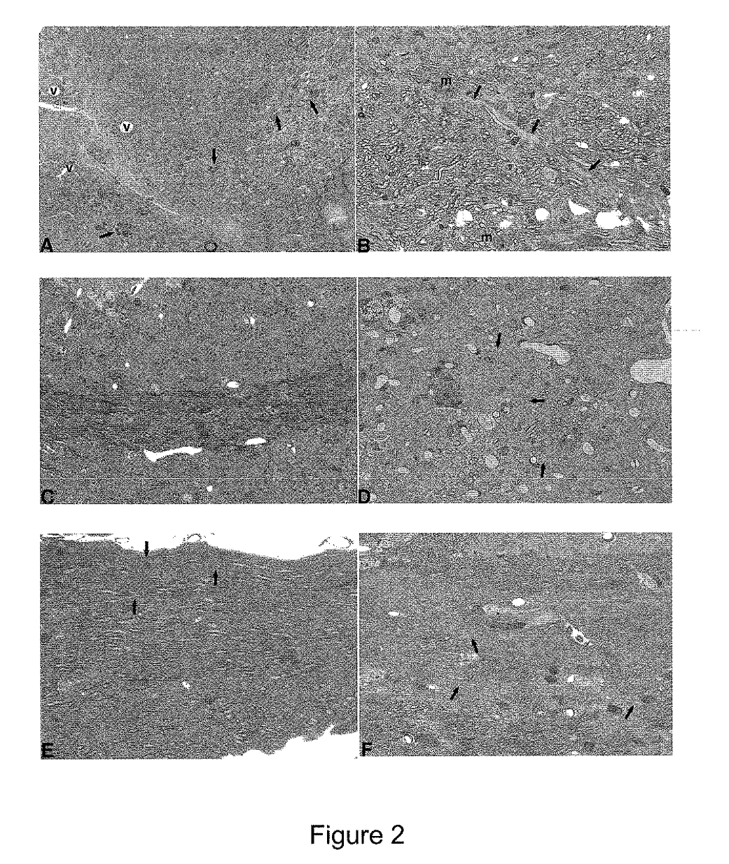

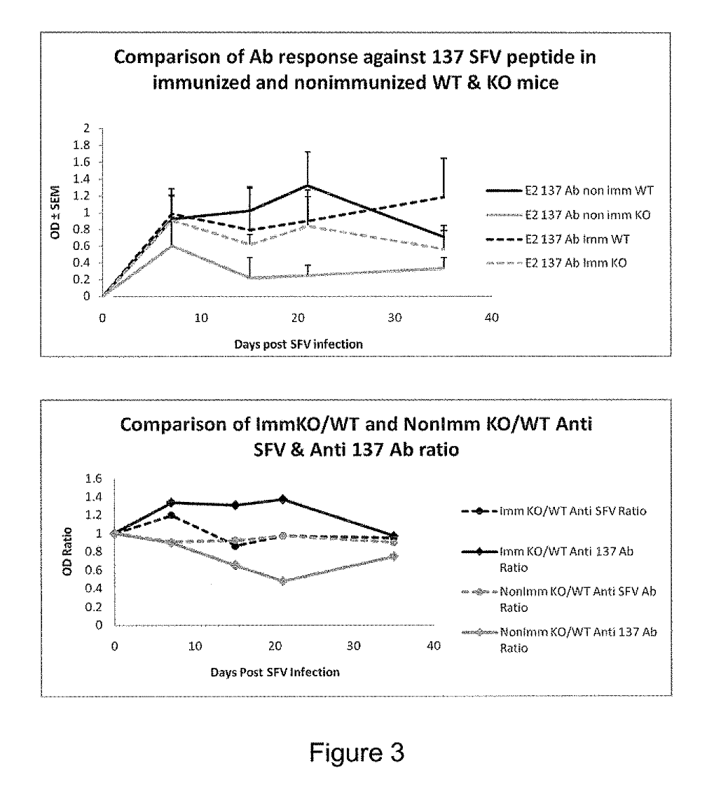

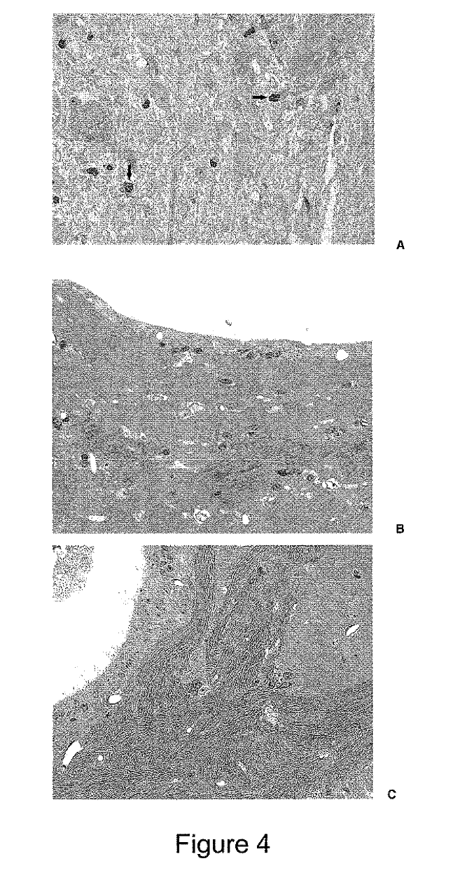

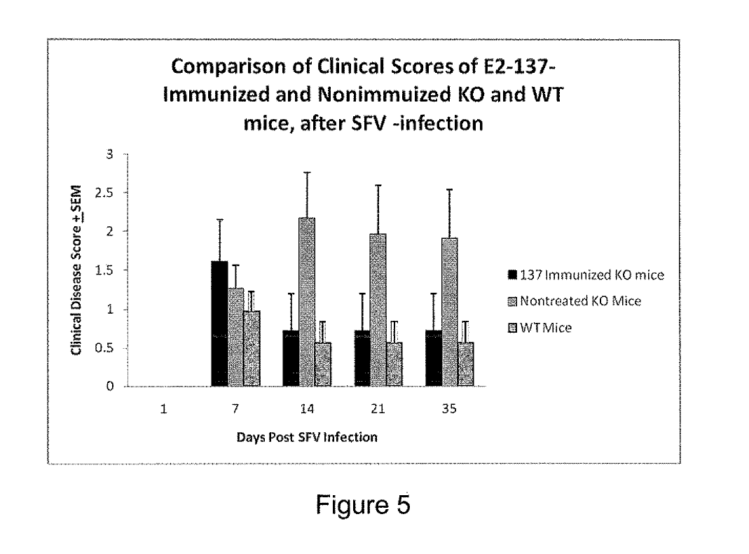

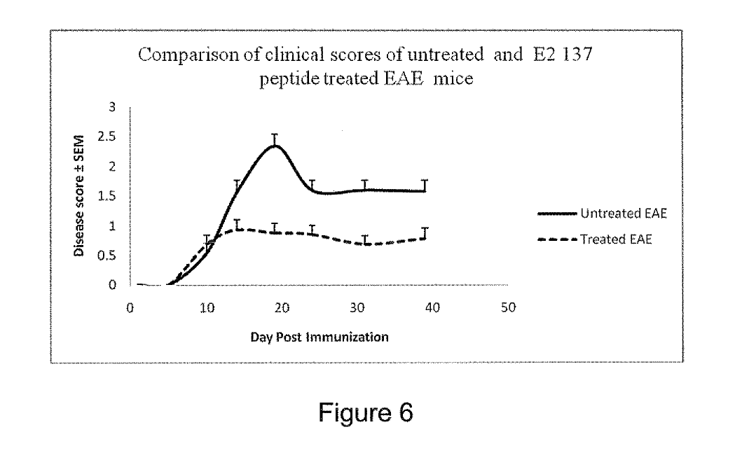

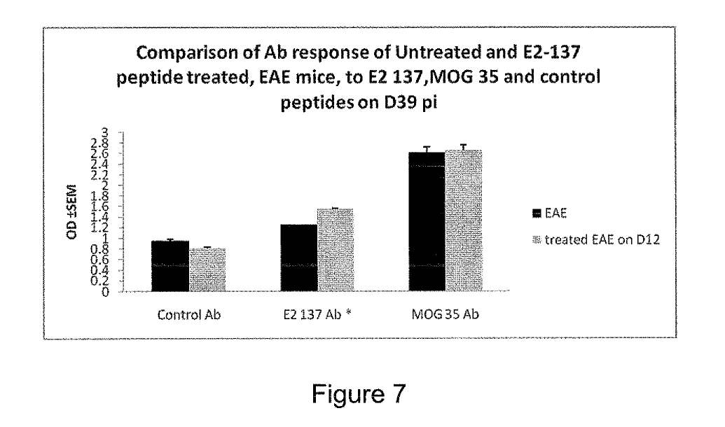

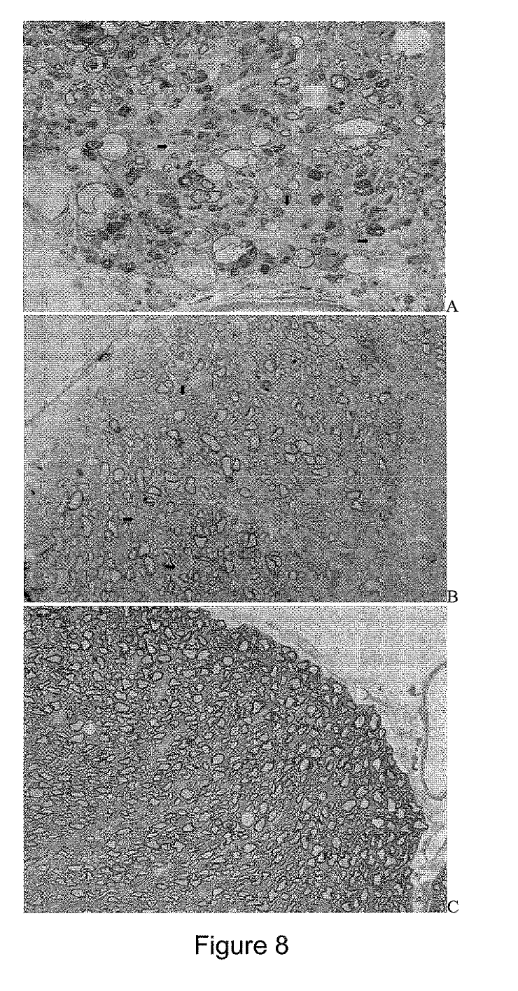

Immunofluorescent staining and flow cytometric analysis was performed as we have previously described (5). Briefly, purified mononuclear cells were resuspended to 10⁵ cells/10 μl in ice-cold FACS buffer supplemented with 10 μg/ml Fc Block (Rat anti-Mouse CD16/CD32 [FcγII/IIIR]) (2.4G2) (BD PharMingen, San Diego, Calif.) and 20 μg/ml mouse IgG2a,κ (Mouse anti-β-2, 6-fructosan, UPC 10) (Sigma) in 96-well microtiter plates. For three-color staining, each well received a 50 μl antibody cocktail containing a 1:100 dilution of the following antibodies: CD3 molecular complex-FITC (17A2, BD PharMingen), CD45R-Tri-Color (RA3-6B2) (Caltag Laboratories, Burlingame, Calif.) for initial gating, and PE-conjugated, TCRγδ (GL3), or TCRαβ (H57-597) (R-PE, BD Pharmingen), and their isotype-matched controls, IgG2_{κ} (B81-3, BD) and IgG2_{λ1} (Ha4/8, BD), respectively. After staining for 45 min. on ice, cells were washed three times in FACS buffer, fixed in 1% Paraformaldehyde (w/v) (Sigma) in PBS and stored overnight at 4° C. Flow cytometry acquisition and analysis was performed, using a FACScan flow cytometer and CellQuest v.3.5 (BD), respectively. Data presented represents 10,000 events. Gating the lymphocyte populations of isolated cells from all mice was performed as previously described (Mokhtarian et al., 2003). The lymphocyte gate, R1, was determined by Forward and Side-Scatter (FSC and SSC), and two-dimensional dot plots were drawn based on the expression of CD3mc and CD45R. Data presented in FIG. 1 are histogram plots of the expression of TCR γδ in the CD3mc⁺CD45R⁻ gate. M1 lines were normalized for background auto fluorescence by unstained cells.

### Preparation of Thin Sections for Demyelination and Remyelination

In order to be able to detect the de- and remyelination accurately, at the end of the experiment, mice will be sacrificed by an overdose of sodium pentobarbital and perfused by intracardiac puncture with Trumps fixative, containing 4% paraformaldehyde and 1% glutaraldehyde. Brains and spinal cords are removed and post fixed in osmium textroxide. Each brain is sectioned coronally into three pieces by cuts through infandibulum and optic chiasm. Spinal cord tissues will be also harvested and processed. Every third block (10-12 blocks per spinal cord, cervical to lumbar range) will be embedded, along with brain sections, in Araldite plastic. The embedded tissues will be cross-sectioned at 1 μm thickness, and the slides stained with toluidine blue or 4% paraphenylenediamine to highlight the myelin sheets. We have studied the one-micron thin sections of the brains of SFV-infected WT and KO on days of 21-42 postinfection. WT mice showed fully-remyelinated white matter by day 35 pi, as has been previously reported by other investigators, including us (5). Sections from brains of SFV-infected KO mice, however, still exhibited either de- or very early remyelination.

Anti-Semliki Forest Virus (anti-SFV) IgG antibodies and normal IgG

The total IgG fraction was commercially prepared (Strategic Biosolutions, Ramona, Calif.) from pooled sera of 3 rabbits immunized once with UV-inactivated SFV (A774 strain) emulsified in CFA and boosted 3 times with antigen in IFA at weeks 3, 5, and 7. The IgG fraction of the pooled antisera was purified by ion-exchange chromatography and contained 8.7 mg IgG/ml of PBS. Antibody activities to SFV and peptides of SFV, to E2, and to recombinant ® MOG and its peptides were confirmed by ELISA. Normal rabbit IgG, isolated from pooled normal rabbit sera by ion-exchange chromatography (Signa Chemical Co., St. Louis, Mo.), was dissolved in saline. Protein concentration was determined by the Bradford assay (Sigma), using normal rabbit IgG as the standard. The IgG solutions were diluted to 3 mg protein/ml in saline and sterilized by passage through a 0.22 μm filter (Millipore Corp., Bedford, Mass.).

### Synthesis of Peptides

The SFV epitopes, E2 peptides 137-151 (NH₂-GREKFTIRPHYGKEI-OH), and 115-129 (NH₂-IQDTRNAVRACRIQYHHD-OH) (SEQ ID NO: 4) were synthesized (Dep. Of Biophysics, JHU, School of Hygiene and Public health, Balto, Md.), as previously described (4) and used for testing antibody responses (5, 9). UV-irradiated SFV A7 (74) virus was used as a positive control, and Purified Protein Derivative (PPD) (Connaught, Swiftwater, Pa.) was used as a negative control.

MOG 35-55 (NH₂-MEVGWYRSPFSRVVHLYRNGK-OH) (SEQ ID NO: 5) was synthesized in the Dept. of Biological Chemistry, Biosynthesis & Sequencing Facility at Johns Hopkins University School of Medicine (Baltimore, Md.).

### Histopathology on SFV-Infected Mice

After transcardial perfusion with ice cold PBS, brains were removed from both WT and γδ KO mice on days 0, 7, 14, 21, and 35 pi and fixed in 10% formalin for histopathology studies. Alternating sections from each brain were stained with hematoxylin and eosin (H&E), and with Luxol fast blue (LFB), as previously described (4), to assess inflammation and demyelination, respectively. Slides stained with H&E were quantified in a blinded fashion for numbers of inflammatory foci (each aggregate containing ≧10 mononuclear cells) for the entire brain. After LFB staining, the prepared slides were observed under the microscope for evidence of demyelination in a blinded fashion, as previously described (4). Slides were graded on the degree of demyelination as follows: 0=no demyelination; 1=rare, scattered areas of demyelination; 2=mild, scattered areas of demyelination; 3=numerous areas of demyelination.

### Brain Pathology Scoring

Mice were sacrificed and perfused by intracardiac puncture with Trumps fixative, containing 4% paraformaldehyde and 1% glutaraldehyde. Brains were removed and post fixed in Trupins. Each brain was sectioned coronally into three pieces by cuts through infandibulum and optic chiasm. The pieces were then dehydrate and embedded in paraffin. Sections from each block were mounted on slides and stained with hematoxylin and cosin to identify pathology in the following brain regions: cortex, corpus callosum, hippocampus, brainstem, striatum, and cerebellum. Pathological scores were assigned without knowledge of the experimental treatment. Each area of the brain was graded as follows:

0=no inflammation
1=minimal inflammation, confined to perivasculature
2=moderate inflammation, including parenchyma infiltration, but no tissue damage
3=intense parenchyma inflammation with minor but definite tissue damage (loss of tissue architecture, cell death, neurophagia, neuronal vacuolation)
4=extensive inflammation and tissue damage thickness).

### Preparation of Thin Sections for Demyelination and Remyelination

In order to be able to detect the de- and remyelination accurately, at the end of the experiment, mice will be sacrificed by an overdose of sodium pentobarbital and perfused by intracardiac puncture with Trumps fixative, containing 4% paraformaldehyde and 1% glutaraldehyde. Brains and spinal cords are removed and post fixed in osmium textroxide. Each brain is sectioned coronally into three pieces by cuts through infandibulum and optic chiasm. Spinal cord tissues will be also harvested and processed. Every third block (10-12 blocks per spinal cord, cervical to lumbar range) will be embedded, along with brain sections, in Araldite plastic. The embedded tissues will be cross-sectioned at 1 μm thickness, and the slides stained with toluidine blue or 4% paraphenylenediamine to highlight the myelin sheets. These lesions were impossible to quantitate due to very small sizes of nerve fibers. Consequently we plan to study the cross sections of spinal cords to quantitate these lesins, as has been previously reported (71). We have previously described these lesions in the spinal cords of SFV-infected mice (5).

### Quantitation of Remyelination of Spinal Cord Lesions

Areas of spinal cord demyelination and remyelination will be determined from multiple cross-sections of plastic-embedded spinal cords, as previously described (71). Using a camera attached to a photomicroscope and an interactive digital analysis system, three parameters will be measured from each side: total white matter area, demyelinated lesion area, and remyelination area. Outline of these regions will be traced and the areas calculated by the computerized digital analysis system. Demyelination will be expressed as the total lesion area as a percentage of total white matter area. Remyelination will be expressed as the total remyelination area as a percentage of the total demyelination lesion area. The criterion for remyelination by oligodendrocytes will be abnormally thin myelin sheaths. All remyelination data refers to oligodendrocyte-mediated remyelination.

### Enzyme-Linked Immunosorbent Assay (ELISA)

A. SFV and Peptides

To measure the antibody response of both WT and γδ KO mice against SFV and two of the peptides of the SFV envelope protein, E2, an indirect ELISA was performed. Sera were pooled from 4-6 SFV-infected WT, and 3 KO, mice on days 0, 7, 14, 21 and 35 pi. Briefly, Immunolon 2HB 96 well microtiter plates (VWR, Bridge Port, N.J.) were coated with 1 μg/well of each antigen in 100 μl/well carbonate buffer overnight at room temperature. After washing, the plates were blocked and serum samples were added. Initial experiments with serial 2-fold dilutions (1:20 through 1:100) of sera against SFV and the two E2 peptides, showed that optimum dilution of the sera was 1:80. After washing, biotinylated goat anti-mouse Igs (BD Pharmingen, Torreyana, Calif.), Strepavidin conjugated to Horse Radish Peroxidase (Vector Laboratories, Burlingame, Calif.), and OPD substrate (Sigma, St. Louis, Mo.) were used, as previously described (4, 9). The difference in absorbance at 490 and 650 nm was measured using a Vmax kinetic microplate reader (Molecular Devises, Sunnyvale, Calif.), and the results expressed as absorbance [OD] units. Values were considered to be positive when they were >0.3 OD units and exceeded the mean value plus three standard deviations for antibody response on day 0 pi of mice of the same strain.

B. Non-Protein Antigens

To assay for anti-recombinant MOG (rMOG) antibody response the wells were each coated with 5 μg/well rMOG (a generous gift from Dr Anne Cross, Washington U, St. Louis) in 100 μl/well carbonate buffer. Sera from above time points pi were added and the assay was performed as above.

Anti-Galactocerebroside (Gal C) IgG production was measured for both WT and γδ KO mice on above days pi by ImmuLisa anti-Gal-C IgG antibody ELISA kit (Immco Diagnostics, Buffalo, N.Y.) with some modifications: Briefly, following overnight incubation with standards, controls and samples (diluted, as directed by the kit), wells were washed 4× and 100 μl/well of cold manufacturer's conjugate (for standards and controls) or 100 μl/well of 1:000 diluted alkaline phosphatase conjugated Horse anti-mouse IgG (H+L) (Vector Laboratories, Burlingame, Calif.) (for samples) was added and incubated for 2 hrs. Following enzyme incubation, wells were washed again and enzyme substrate was added for 30 min. The plates were read at 405-650 nm and results were expressed in Enzyme Units/ml, according to the standard curve. An average±SD of three experiments is shown for each day post infection.

### Statistics

Clinical observations, histopathological results and anti Gal-C and SFV epitope antibody responses (ELISA) were analyzed using a two-tailed, and rMOG ELISA data were analyzed using a one-tailed Student's t-test, by InStat v.2.01. KS statistics were performed for flow cytometry data on Cell Quest v.3.5.

The effect of Anti E2 137-151 Antibody in Remyelination

A. Low Doses of E2 137 Immunization During SFV Disease

WT and KO mice were immunized subcutaneously with E2 137 (0.5 mg/injection or 25 mg/kg, based on the average weight of 20 g/mouse) in IFA or IFA alone on day one pi. Additional injections of either E2 137/IFA, IFA alone or PBS alone, were performed on days 5, 10, and 15 pi. Mice were sacrificed on day 35 pi and spinal cord demyelination and remyelination pathology will be measured.

B. High Doses of E2 137 Immunization During SFV Disease

Single or multiple injections of E2 137, at 1 mg/injection will also be used. SFV-infected WT and KO mice will be immunized subcutaneously with 1 mg of E2 137 (50 mg/kg, based on the average weight of 20 g/mouse) in IFA, or IFA alone, on above days. Additional injections of either E2 137, or PBS, in IFA, will be performed as above. Mice will be sacrificed on day 50 and spinal cord demyelination and remyelination pathology will be measured, as above.

C. Passive transfer of E2 137-151 antibody.

All antibodies will be dissolved in PBS and administered intraperitoneally. Normal control mice will be given protein G-purified antibodies isolated from commercially purchased mouse serum. These will be used as a control for antibodies against E2 137.

A first group of mice will receive 0.1 mg purified anti E2 137 antibody intraperitoneally by 4-8 injections from day 8 pi, administered weekly or at 4-day intervals, for a total of 0.8 mg of antibody, over 35 days. The second group of mice will receive normal antibody, administered similarly in injections of 0.1 mg each during 35 days. Mice will be sacrificed on day 45 pi and in all groups spinal cord demyelination and remyelination will be measured after sacrifice. The third group of mice well receive 1.2 mg of antibody against E2 137-151, administered in 4 injections with weekly over 35 days. PBS will be administered to a control group of mice.

### Serum Isolation

Mice were bled 5 times between days 14-45 pi. After each bleed, blood were stored overnight at 4° C., and then centrifuged to isolate serum. Serum was stored at −20° C. until all bleeds were completed.

### Immunohistochemistry on Cultured Cells

Glial cultures (mixed or oligodendrocytes-enriched) will be derived from cerebral hemispheres from 4-7 day old Sprague-Dawley rat pups (Harlan Sprague Dawley. Indianapolis, Iowa), maintained on poly-lysine-coated glass coverslips in DMEM medium containing 10% fetal bovine serum, and immunostained between Days 4-28 in vitro. CNS glial cultures will also be derived from adult human brain biopsies (obtained from surgical correction of epilepsy). Mouse peritoneal macrophages will be derived by lavage, 5-8 days following intraperitoneal injection of sterile, 3% thioglycolate solution and maintained in RPMI medium containing 5% fetal bovine serum for 1-3 weeks. Spinal cord sections will be obtained by cryostat sectioning of frozen spinal cords (10 μm thickness). Sections will be lightly fixed in ice-cold 95% ethanol for 5 min and incubated in 10% goat serum to reduce nonspecific staining.

Application of primary antibodies in PBS buffer will be performed with ice-cold solutions with culture plate on ice with the intention of staining the cell surface. Primary antibodies will be applied for 30-45 min. After rinsing in PBS for 10 min, fluorophone-conjugated secondary antibodies diluted in ice-cold PBS will be applied for 30 min. Cells will be then rinsed with PBS for 10-15 min. Fixation with 4% paraformaldehyde occurred either once, following the final PBS rinse, or twice, just prior to secondary antibody application and following the final PNS rinse. Cells will be viewed with Olympus fluorescent microscopes.

The primary antibodies will include antibodies against E2 137-151 and MBP 56-68 (4-40 ug/ml) Normal antibodies (20 ug/ml), anti glial fibrillary acidic protein (GFAP, an astrocyte marker) (Dako, Carpinteria, Calif.), 01 (mature oligodendrocyte marker) 04 (oligodendrocyte marker) A2B5 (immature oligodendrocyte marker) 94.03 (oligodendrocyte marker) isolectin B₄, CD11b (complement receptor 3)(activated microglia and macrophage markers), rat anti-F4/80 (Serotec, Raleigh, N.C.), biotinylated rat anti-Fcy III/II receptor (CD16/CD32, BD PharMingen, San Di ego, Calif), rat anti-myelin basic protein (82-87; Calbiochem, San Diego, Calif.). The secondary antibodies will be anti-species IgG or IgM, raised in goat, and fluorophore-conjugated (Jackson Immunoresearch; Vector) for direct detection or biotinylated for detection by the peroxidase method using an ABC Elite kit (Vector).

Antibodies and normal mouse antibodies will be usually applied as biotinylated derivatives. Biotinylation was performed by 30 min incubation of purified antibodies with EX-link NHS-LC biotin (Pierce), followed by extensive dialysis against PBS (10,000 molecular weight cutoff). Western blot and ELISA will confirm Biotinylation and preservation of binding activity.

### Example 1

### Analysis of TCR γδ⁺Cell Surface Markers of SFV-Infected WT and γδKO by FA CS Confirms the Scarcity of CD3⁺TCRγδ⁺ T Cells in KO Mice

A. TCRγδ

It has been previously reported that IEL are a rich source of CD3⁺TCRγδ⁺ T cells (70), and thus were selected as a positive control for this experiment. To ensure that the γδ KO mice lacked TCRγδ⁺ T cells, IEL were purified from the small intestines of SFV-infected animals along with the BMNC and SMNC. Using the isotype-matched control antibody, the percentage of TCR γδ in the CD3⁺CD45R⁻ gate was %1, in day 0 mice, and was <%1 for other days pi. The percentage of TCRγδ⁺ BMNC cells from non-infected WT mice in the CD3⁺CD45R⁻ gate, using GL3 antibody, was 3.3% on day 0 pi, in experiment 3, shown here as representative (FIG. 1A, top row). After SFV-infection, on day 7 pi, the percentage of TCRγδ⁺ BMC, increased to 6.2%, decreased on day 14 pi to 4.8%, and continued to decrease to 1.3% on day 21 pi, and was 2.1% on day 35 pi (FIG. 2 A). Due to the unavailability of a large number of γδ K10 mice for each experiment, their cells were only harvested on day 21 pi. BMNC from γδ KO mice did not express detectable levels (<1%) of TCRγδ marker on their T cells (FIG. 1B).

TCR αβ cells in the KO group, as in WT, were 47.1% of the lymphocytes (R1) and >99% of T-cells, in SPMNC, as expected (data not shown).

B. TCRαβ

As a control, changes in TCRαβ T cells were also determined along with TCR γδ T cells, by FACS. In SFV-infected WT mice, TCRαβ marker was expressed on the CD3⁺ cells of BMNC, at greater than 90% on all days tested, and its fluctuations closely followed that of CD3⁺ T-cells. TCR αβ⁺ cells were also >90% of the CD3⁺ cells in SPMNC, on all days pi days tested. Although a significant number of TCR αβ⁺ cells (up to 40%) were found in the IEL preparation, these cells could have come primarily from the lamina propria and may be present in IEL preparations as contaminants, as also reported by others (70).

TCR αβ cells in the KO group, as in WT, were 47.1% of the lymphocytes (R1) and >99% of T-cells, in SPMNC, as expected (data not shown).

Clinical Disease in SFV-Infected γδ KO is More Severe than in WT Mice

SFV-infection of B6 and γδ KO mice produced three types of clinical outcomes (1) weakness, ruffled fur, and weight loss, from which the mice may recover or progress to (a) permanent paralysis and (b) death, as previously described (4). Mice were observed daily for clinical manifestations of SFV and were scored on a scale of 0-6, as follows: 0=no abnormality, 1=mild hind limb weakness (some difficulty righting themselves when turned on their back), 2=moderate hind limb weakness, sometimes associated with floppy tail, 3=hind limb paresis, accompanied by some forelimb weakness, sometimes more marked on one limb or one side, but not complete paralysis, 4=complete paralysis of hind limbs, accompanied by mild forelimb weakness 5=paralysis of hind limbs, associated with moderate forelimb weakness, and 6=quadriplegia, moribund (leads to death).

Normally in SFV-infection, B6 mice show acute sings of disease on days 6-8 pi, and start to recover after that. It should be noted, that stages 1-3 are weakness primarily due to systemic viral effects from which mice recover, and stages 4-6 are permanent and finally fatal paralysis due to CNS immunopathology (4).

In a pool of three experiments, WT mice did not display signs of illness on day 4 pi, while three γδ KO mice showed symptoms on this day (Table 1). The KO group showed a significantly higher average clinical score than the WT group on day 7 pi (2.4 vs. 1.6). (p<0.01). Mice in both groups recovered (either partially or completely) following day 7. As a result the clinical scores of WT and KO mice were not significantly different on the following days pi (Table I). Some sick mice were harvested, especially on day 7 pi, for virological, histological and flow cytometric studies, resulting in a decrease in their number on different days.

### SFV-Induced Inflammation and Demyelination are Not Different in γδKO and WT Mice

Inflammation in the brains of SFV-infected mice was noted on day 7 pi in both groups. At this time, perivascular and parenchymal inflammation were widespread, characterized by focal lymphocytic infiltrates around endothelial cells, and occasional vacuolation of parenchyma, and eventually led to gray and white matter pathology throughout the cerebrum and the cerebellum, as has previously been reported (9). For both groups, the peak of the inflammatory changes was on day 7 pi. Inflammatory foci were spread throughout most areas of the brain, namely, cerebral hemispheres, cerebellum and brainstem (average number of inflammatory foci was 20.6 in WT and 24.0 in KO mice). Numbers of focal inflammatory lesions at day 7 post-infection in γδ KO mice, though higher, were not significantly different from WT mice (Table II). The inflammatory response was reduced on days 14 and 21 pi in both groups, and then diminished in WT, but only slightly decreased in γδ KO mice, on day 35 pi, (average number of inflammatory foci was 1.6 in WT and 4.0 in KO mice, in three separate experiments.

Staining with LFB (taken from the same brain as the samples stained for H&E) was performed and analyzed separately for the cerebellum and the rest of the brain. In the brain parenchyma, vacuolation accompanied by inflammatory cells in the same area was observed in both WT and γδ KO mice on day 7 pi. On days 14 and 21 pi, areas showing loss of myelin, in the absence of inflammatory cells were observed, especially in the cerebellum, on day 21 pi, in WT and KO mice. By day 35 pi, the WT sections showed intense blue staining especially evident in the cerebellum that appeared fully remyelinated. The brain sections of γδ KO mice, however, still exhibited numerous areas of vacuolation and were not remyelinated. Because the demyelinating effects of SFV primarily affects the cerebellar white matter (Mokhtarian and Swoveland, 1987), the demyelination (vacuolation scores), when assessing the cerebellum exclusively, yielded more striking results (Figure not shown). The largest average vacuolation/demyelination score averaged from three experiments occurred on day 21 pi, in WT mice (2.3) and in the γδ KO group (2.17) (Table III).

Virus Replication in SFV-Infected γδ KO is Higher than in WT Mice

Viral titers were determined in three experiments. No brains from non-infected, day 0, mice, in either group, had any detectable virus by our method. Viral replication was detected on day 3 pi in brains from SFV-infected WT and KO mice, and reached an average peak titer of 10⁴ on day 5 pi (Table IV). The WT mice had cleared the virus to non-detectable levels, by day 7 pi, while the γδ KO did not completely clear the virus until day 14 pi (Table IV) and had higher viral titers in their brains than WT mice during the second week of infection.

### Example 2

### Remyelination is Suppressed in SFV-Infected γδ-KO, Compared to WT Mice

It should be noted that demyelination occurs after SFV-infection and is immune-mediated (4). It has been shown that SFV-infection of T-cell deficient, Nude mice (2, 3) and B-cell deficient mice do not lead to demyelination and results in persistence of virus replication. Therefore, immune responses and specifically antibody response, and not virus, are the factors that cause the demyelination (2, 4).

We have previously reported (5) that remyelination follows the demyelination soon after it occurs. In 1 micron thin sections, layers of thin myelin are evident in demyelinated areas, mixed with naked axons, soon after demyelination is widespread in the cerebellum (5) on days 15-21 pi. Remyelination is complete by day 35 pi, in wild-type B6 mice.

Similarly, in this study, by day 35 pi, the WT sections appeared fully remyelinated, which was especially evident in the cerebellum. The brain sections of γδ KO mice, however, still exhibited numerous areas of vacuolation and were not remyelinated. The average vacuolation/demyelination score from three different experiments was 1.6 in KO mice, which was significantly higher than WT mice (p<0.05).

### Remyelination

In order to be able to analyze the demyelination and especially remyelination accurately, we then studied the one micron thin sections of the brains of SFV-infected WT and KO mice on days 7, 14, 21, 35 and 42 postinfection. Inflammation and demyelination is seen on days 14-21 pi in both WT and KO mice (FIGS. 2 A&B), shown as representative photomicrographs. CNS white matter of WT mice was fully remyelinated by day 35 pi, as has been previously reported by us (5) and many other investigators (2) (FIG. 2 C) and remained so on day 42 pi (FIG. 2 D) (shown for comparison with KO mice (2E and 2F). Unlike WT, sections from brains of KO mice, however, exhibited many fibers that were either unmyelinated or were at very early remyelinating stages, on days 35, as shown by an arrow (FIG. 21). The brains of KO mice were still not fully remyelinated even at 42 days pi, where lightly stained and darkly stained areas of very early remyelination (arrow) and demyelinated regions are alternating. Note that the nerve fibers are longitudinally orientated in corpus callusum (FIG. 2F).

### Example 3

### Antibody Responses of γδ KO to SFV Epitope, 137-151 is Lower than WT Mice

Sera obtained from SFV-infected WT mice, were used at a pre-determined dilution found in the initial serial dilution curves of sera (normally 1:80) with SFV and the two E2 epitopes, in ELISA assays. The sera reacted strongly with SFV and with E2 137-151 epitope (FIG. 3), on day 7 pi, increasing in titer on days 14, 21 and 35 pi, in three separate experiments. Although sera from SFV-infected γδ KO mice reacted vigorously with SFV, they marginally reacted with E2 137-151. The reactivity of KO sera with this epitope was less than with WT sera on all days pi (FIG. 3), and at significantly lower levels during the peaks of antibody response and remyelination on day 21 and day 35 pi (p<0.01 and <0.05, respectively).

The reactivity of sera from SFV-infected KO mice with E2 115-129 is also shown in FIG. 3. These antibody responses were lower, but not significantly different than WT, on all days pi. Sera from neither groups showed reactivity with control protein, Purified Protein Derivative (PPD) (FIG. 3).

Responses to Non-Protein Components of Brain are Similar in both WT and KO Groups

Sera from SFV-infected WT mice reacted to rMOG on day 7 to 35 pi, and was significantly higher than day 0 (p<0.05 vs. day 0). Sera from KO mice also reacted to rMOG on days 7 to 35 pi, and was significantly higher than day 0 (p<0.001). In both, the anti-rMOG antibody response was twice as the background level and remained constant during the entire experiment (data not shown). Overall, however, no significant differences were seen between the sera from WT and KO mice in their anti-rMOG antibody responses. The reactivity of these sera with rMOG was stronger than with the control protein, PPD, on all days pi and the PPD reactivity of SFV-infected sera never reached statistically significant higher levels than day 0 sera, on any days pi.

Our studies of SFV-infection of mice, lacking TCR γδ⁺ T cells (KO), have shown that the usual complete remyelination and production of antibody to one of the surface viral epitopes of SFV are both decreased in these mice. Although γδ⁺ T-cells respond to lipids and non-protein antigens, to our surprise, reactivity to non-protein cell surface component, Gal-C, and to rMOG, were equally high in both, SFV-infected WT and KO mice. Similarly, inflammation, and antibody production to SFV and to another epitope of SFV(E2 115-131), were not different in the two groups. We conclude that antibody production to the surface epitope of SFV, E2 137-151, is the mediator of remyelination and repair of CNS following SFV-infection.

### Example 4

### Homologies Between Epitopes of Sfv and Proteins of Myelin

Our previous studies have revealed amino acid (aa) homologies (mimicry) between the E2 peptides of SFV and some of the peptides of myelin (MOG and MBP) (data not shown). E2 is the major surface glycoprotein of SFV containing the T cell epitopes in the region of aa 115-151 (63, 64). Only areas of 3 consecutive complete homologies (combined with some partial) considered as significant homologies. The aa sequences containing the mimicked areas were aligned again to further verify their molecular mimicry (Table V). To improve antigenicity, a slightly longer form of these peptides: E2 115-131 and E2 137-151, and their matching peptides in myelin proteins, MOG 18-32 (47-58 of the longer form, ref.21-23), PLP 89 104, MBP 56-68 and MBP 64-75 were synthesized. Fibrinopeptide B, 1-14 and Fibroblast growth factor, 106-120 (FB and FGF), (Sigma) were used as negative control peptides (Table V). We then found that lymphocytes of SFV-infected mice cross-proliferated to these mimicked peptides and immunization with E2 115-131, but not with E2 137-151, induced an autoimmune like disease in B6 mice. Thus we have shown that autoimmunity may develop when peptides of a virus like SFV have only a short sequence homology with host self peptides.

As stated above we also found significant homology between the amino acid sequence of E2 137-151 peptide and its mimicked peptide, MBP 56-68. These mimicked peptides also demonstrated cross recognition by antibody response (Mokhtarian et al 1999).

### Example 5

### Evidence of Molecular Mimicry with Mouse and Human MBP

We have previously found the presence of antibodies reactive with MBP and MOG, in SFV-infected mice. These responses occurred concomitantly with the responses to SFV and E2 peptides. E2 is the major surface glycoprotein of SFV containing the T cell epitopes in the region of 115-151 (4). This finding led to our previous studies of molecular mimicry between SFV E2 and peptides of MBP, PLP and MOG. Significant homologies existed between the regions of E2 115-129 and MOG (aa18-32 of short form). Moreover, we found homology between the amino acid sequence of E2 137-151 epitope of SFV and a peptide of mouse MBP (aa 56-68) and human MBP, aa 102-118, as shown below. Mice inoculated with E2 115-129 induced vaccuolation in myelin (4), whereas similar inoculation with E2 137-151 did not cause histopathology in mice (unpublished data). Previous pepscan studies of E2 137-151 have shown that the three amino acid HYG is the important part of E2 137-151 for T cell help (72). Thus, short copolymers of HYG, such as HYGH (SEQ ID NO: 6), HYGHG (SEQ ID NO: 7), HYGHYG (SEQ ID NO: 8), HYGHYGH (SEQ ID NO: 9), HYGHYGHY (SEQ ID NO: 10), HYGHYGHYG (SEQ ID NO: 11), HYGHYGHYGH (SEQ ID NO: 12), HYGHYGHYGHY (SEQ ID NO: 13), HYGHYGHYGHYG (SEQ ID NO: 14), HYGHYGHYGHYGH (SEQ ID NO: 15), HYGHYGHYGHYGHY (SEQ ID NO: 16) and HYGHYGHYGHYGHYG (SEQ ID NO: 17) are contemplated by the present invention. Additional copolymers of GRE such as GREG (SEQ ID NO: 18), GREGR (SEQ ID NO: 19), GREGRE (SEQ ID NO: 20), GREGREG (SEQ ID NO: 21), GREGREGR (SEQ ID NO: 22), GREGREGRE (SEQ ID NO: 23), GREGREGREG (SEQ ID NO: 24), GREGREGREGR (SEQ ID NO: 25), GREGREGREGRE (SEQ ID NO: 26), GREGREGREGREG (SEQ ID NO: 27), GREGREGREGREGR (SEQ ID NO: 28) and GREGREGREGREGRE (SEQ ID NO: 29) are contemplated by the present invention. Further copolymers of GKH such as GKHG (SEQ ID NO: 30), GKHGK (SEQ ID NO: 31), GKHGKH (SEQ ID NO: 32), GKHGKHG (SEQ ID NO: 33), GKHGKHGK (SEQ ID NO: 34), GKHGKHGKH (SEQ ID NO: 35), GKHGKHGKHG (SEQ ID NO: 36), GKHGKHGKHGK (SEQ ID NO: 37), GKHGKHKHGKH (SEQ ID NO: 38), GKHGKHGKHGKHG (SEQ ID NO: 39), GKHGKHGKHGKHGK (SEQ ID NO: 40) and GKHGKHGKHGKHGKH (SEQ ID NO: 41) are contemplated in the present invention.

| | |
|---|---|
| SFV E2 137-151 | GREKFTIRPHYGKEI |
| & | : ...:. :::. |
| Mouse MBP 56-68 | GKDSHTRTTHYGS |
| SFV E2 137-151 | GREK--FTIRPHYGKEI |
| & | ::: : :: . :. |
| Human MEP | GREDNTFKDRPSESDEL |

Two dots indicate complete and one dot is indicative of partial homology.

### Example 6

### Antibody Responses During SFV Infection

Table VI shows the differences between the OD's of day 0 and different days after infection. Sera from SFV-infected B6 mice reacted from day 7 on pi, in ELISA assays, with SFV proteins (UV-SFV) (p<0.03) and on later days with E2 137-151 (p<0.05) and E2 115 129 peptides (p<0.02). The sera reacted significantly with 1E2 115-129-mimicked peptide, MOG, 18-32 (p<0.005, d14) and PLP 89-104 (p=0.005). Similarly, The sera reacted significantly with E2 137-151-mimicked peptide, MBP 56-68 (p<0.005). MBP 56-68 shares aa HYG (3 aa homology) with E2 137-151 (see Table I, above.

The sera of SFV-infected B6 mice also reacted significantly with MOG and MBP. In some instances day 0 OD values were high, thus making the differences appear small. Therefore, to account for such discrepancy, we also determined the ratio of the highest day (day 15 or 21) over day 0. This indicated that MOG 35 had a high ratio because of very low day 0 OD value and MBP had a low ratio because of high day 0 OD value (due to its stickiness).

Establishment of Molecular Mimicry as a Cause of Cross-Reaction of SFV-Infected Sera with Myelin Peptides

As seen in Table VII, the reactivity of these antibodies was then compared with their molecular mimicry data. We have found that peptides of myelin with highest percentage of mimicry with peptides of SFV, E2 (shown in italics) also show highest Ratio AND highest difference in their reactivity with the corresponding antigen in ELISA assay.

Since infection with live SFV, like that of Theilers virus, could cause epitope spreading, one could argue that responses to other myelin peptides, like MOG 18-32, is also due to epitope spreading.

### Role of Molecular Mimicry in Antibody Response is Truly Shown in Immunized Rabbits

To prove molecular mimicry between E2 137-151 and MBP 56-68 and between E2 115-131 and MOG 18-32, we performed the following experiment: Using a polyclonal rabbit serum prepared against UV-inactivated SFV antigens, we performed ELISA with peptides and proteins of myelin and SFV E2, as above.

In this experiment, the IgG fraction of serum from rabbits immunized with UV-inactivated SFV, in which case epitope spreading does not occur, shows strong immunoreactivity to this antigen and to two peptides of the major viral coat glycoprotein E2 (E2₁₁₅₋₁₃₁ and E2₁₃₇₋₁₅₁) by ELISA. (Table VIII) Cross-reactivity to the peptides MOG₁₈₋₃₂ and MBP₅₆₋₆₈ that have molecular mimicry with E2₁₁₅₋₁₃₁ and E2₁₃₇₋₁₅₁, respectively, were also demonstrated, farther confirming the existence of molecular mimicry between these peptides.

### Quantitation of Anti-SFV Antibody Response (OD Results)

The polyclonal rabbit anti SFV antibody was commercially prepared (Strategic Biosolutions, Ramona, Calif.) from pooled sera of 3 rabbits, immunized once with UV-inactivated SFV (A774 strain) emulsified in CFA and boosted 3 times with antigen in IFA at weeks 3, 5, and 7.

The total IgG fraction of the pooled antisera was purified by ion-exchange chromatography and contained 8.7 mg IgG/ml of PBS. This antibody reacted to SFV (and to E2 peptides) and will be used for quantitative studies of antibodies to SFV and to peptides of SFV (Table IX).

### Example 7

### The Positive effect of Anti EZ 137-151 Antibody Production in Remyelination by Treatment with

A. E2 137-151 Peptide Immunization During Sfv Disease

It has been shown that TCR αβ⁺ T cells can perform in the absence of TCRγδ⁺ T cells in some conditions (45). Therefore, we attempted to increase the titer of anti E2 137-151 antibody by immunization of δKG mice with this peptide.

WT and KO mice were immunized subcutaneously with E2 137-151 (0.5 mg/injection or 25 mg/kg, based on the average weight of 20 g/mouse) in IFA on the day of SFV-infection. Additional injections of E2 137/IFA were performed on days 5 and 15 for WT and KO mice. Mice are sacrificed on days 15, 21, 35 and 42 pi and sera were tested by ELISA for reactivity with SFV and its peptides. As reported in FIG. 3, this time the KO mice were able to make a better antibody response to E2 137-151 than the WT did (FIG. 3A). This immunization, however, appeared to have suppressed the formation of anti-E2 137-151 antibody in SFV-infected, E2 137-151-immunized WT mice. We then determined the ratio of antibody production to SFV and to E2 137-151, in immunized versus non-immunized KO and WT mice and found that while this immunization did not increase the ratio of antibody production to SFV, it significantly increased the ratio of KO/WT anti E2 137-151 antibody production (FIG. 3B).

Furthermore the preliminary studies indicated that suppression of anti E2 137-151 antibody in SFV-infected WT mice delayed the clearance of virus from the brains of WT mice and increased the clinical signs of viral encephalomyelitis.

Histopathological studies of E2 137-151 peptide-immunization of SFV-infected WT and KO mice showed abundant number of plasma cells in the brains of peptide-immunized SEV-infected KO and WT mice (FIG. 4A, Arrow). Peptide immunization resulted in early remyelination (Arrow) on day 28 pi, in KO mice (FIG. 4B) and normal remyelination in WT mice. A normally myelinated tissue is shown for comparison (4C).

B. Passive Transfer of E2 137 Antibody in SFV-Infected Mice.

To further study the mechanism of induction of the proposed remyelinating antibodies, polyclonal and monoclonal antibody (Mab) will be produced to E2 137-151 (and to the mimicked MBP 56-68 or 64-75). We have previously prepared B cell hybridomas from spleens of SFV-infected mice, which are responsive to E2 137-151. These responses were not entirely monoclonal: B cell clones responding to SFV epitopes also reacted with the mimicked peptides of each clone. While monospecific B cell clones responding to the mimicked myelin peptides were present, no monospecific clone to SFV epitopes were found.

In this study, in order to generate these remyelinating antibodies mice will be immunized with different doses of E2-137-151 peptide. Polyclonal and monoclonal anti E2-137-151 (and MBP 56-68) antibodies will be prepared from spleens of immunized mice, as previously described (Strategic Biosolutions, Newark, Del.). These antibodies (500 μg/mouse in 0.5 ml) will be passively transferred by ip injection, into 8-10 γδ KO B6 recipients, infected with SFV, 8-14 days earlier. We expect to see remyelination in otherwise demyelinated brains of these mice. Controls will be injected with the same amount of anti E2 115-129 antibody. As above, three mice from each of the recipient groups will be sacrificed 15 days after adoptive transfer for histological and immunological determinations (see above). The remaining 5-7 mice will be sacrificed later at the termination of experiment for similar evaluation and for ELISA assays.

### Example 8

### The Suppressive Effect of E2 137-151 Peptide Immunization on Clinical Scores of SFV-Infected TCR-γδ-KO, Compared to WT Mice

Method—Five-week old C57B16 (B6) WT and KO mice (Jackson Laboratories, Bar Harbor, Mass.) were infected with SFV, and KO mice were immunized subcutaneously with E2-137-151 (0.5 mg/injection or 25 mg/kg, based on the average weight of 20 g/mouse) in IFA, one day after SFV-infection. Additional inoculations of E2 137-151/IFA were performed on days 5 and 15 for KO mice only. Animals were observed daily for clinical manifestations of disease and were scored on a scale of 0-VI. 0=no abnormality; I-mild hind limb weakness (some difficulty righting themselves when turned on their back); II=moderate hind limb weakness, sometimes associated with floppy tail; III=weakness of hind limbs accompanied by some forelimb weakness, sometimes more marked on one limb or one side, but not complete paralysis; IV=hind limb paresis accompanied by mild forelimb weakness; V=paralysis of hind limbs, associated with moderate forelimb weakness; and VI=quadriplegia, moribund.

Result—The average severity of the disease decreased significantly in E2-137-151-immunized KO compared with non-immunized KO mice (p value<0.001) (FIG. 5). Peak of the disease in Untreated KO mice was between day 10-12 however in WT group disease peak was between day 6-7, so disease started later with more severity (FIG. 5). This result showing improvement of clinical scores in E2 137-151 immunized KO mice, is in line with the antibody results; showing that peptide immunization led to increase of anti E2 137-151 antibody in SFV-infected KO mice and remyelination.

### Example 9

### Using EAE as a Model of MS Studies of TCRγδ⁺ Cells in EAE

To induce EAE, 7-8 wk old B6 mice (Jackson Laboratories, Bar Harbor, Mass.) were each inoculated with 300 μg MOG 35-55 in Complete Freund's Adjuvant (CFA) and CFA only, as we have previously reported (4). Mice were evaluated daily for clinical signs of disease and severity was assessed on a scale of 0-6 (4, 20). A severe clinical EAE was induced in MOG/CFA inoculated mice, and reached a peak on day 17 pi, the ACS of which was 2.85. On day 25 pi, the ACS of EAE animals, for all days, was 2.15. At the end of the experiment, on day 42 pi, EAR mice had ACS of 1.41. The average clinical severity of all mice (ACS) of normal control and the B6 mice inoculated with CFA only remained unchanged all through the 42 days of observation.

### FACS Studies

### Expression of TCRγδ on BMNC of MOG 35-55 Induced EAE Mice is Low During Acute Phase and Increases During Chronic Phase

Four to five WT mice that developed EAE were sacrificed during the acute (day 15 pi) and chronic (day 42 pi) phases of the disease to determine the expression of TCRγδ on BMNC by FACS. Mice immunized with only the CFA adjuvant were used as a negative control. BMNC were purified and used in CD3/CD45R staining as described above for SFV-infected mice.

At the time of acute EAE in B6 mice, mice immunized with only CFA had less than %1 TCRγδ⁺ T-cells in total lymphocyte gate of BMNC. This comprised of 9.1% TCRγδ⁺ T-cells in the T cell gate (CD3⁺CD45R⁻). At the time of acute EAE in B6 mice, TCRγδ⁺ T-cells in BMNC increased to 1.8% of the total lymphocytes; and 7.2% of the T-cells (data not shown).

During the chronic phase of the disease (day 42 pi) TCRγδ⁺ T-cells increased in percentage to 20.1% of all T-cells, and were 2.4% of the lymphocyte gate.

In conclusion, unlike in SFV-infection, in acute EAE, TCRγδ⁺ T cells were fewer initially during the acute inflammation and increased later, both in intensity and proportion, during chronic stage. This may suggest their involvement in the recovery period of EAE.

### Example 10

### The Suppressive Effect of E2 137-151 Peptide Treatment on Clinical Scores of E2-137-151-Treated EAE Mice, Compared to Non-Immunized EAE and Control (CFA Only) Groups

Method—To induce EAE, fourteen 7-8 wk old B6 mice (Jackson Laboratories, Bar Harbor, Mass.) were each inoculated, subcutaneously, with 300 mg MOG 35-55 in Complete Freund's Adjuvant (CFA) or CFA only (as control), followed by two injections of 100 ng of pertusis toxin, 48 hours apart (9, 73). Eight mice in EAE-induced group were immunized subcutaneously with E2-137-151 (0.5 mg/injection or 25 mg/kg, based on the average weight of 20 g/mouse) in IFA, one day after MOG inoculation. One additional inoculation of E2 137-151/IFA was performed 5 days later. Animals were observed daily for clinical manifestations of disease and were scored on a scale of 0-VI. 0=no abnormality; I=mild hind limb weakness (some difficulty righting themselves when turned on their back); II=moderate hind limb weakness, sometimes associated with floppy tail; III=weakness of hind limbs accompanied by some forelimb weakness, sometimes more marked on one limb or one side, but not complete paralysis; IV=hind limb paresis accompanied by mild forelimb weakness; V=paralysis of hind limbs, associated with moderate forelimb weakness; and VI-quadriplegia, moribund.

Result—Mice were immunized with MOG 35-55 in CFA, to induce EAE as described above. In the first two experiments, mice were divided into two groups, untreated, and treated after EAE induction. The treated EAE group received E2-137-151 peptide in IFA, subcutaneously on D5 post-EAE induction, before the signs of clinical disease were apparent. This method resulted in the absence of clinical signs of EAE in the E2 137-treated RAE group, which made our interpretation of treatment unclear.

Next, three experiments were performed with a total number of 21 RAE and 22 late treated EAE mice. The EAR induced group was treated on D12 post-EAE induction when they exhibited clear clinical signs of EAE. EAE mice were further treated with E2-137-151 peptide on days 17 and 22 post-EAE induction. Disease severity in EAE mice was significantly higher than the EAE mice treated on D12. Peak of the disease in both RAE and D12 treated EAR mice were on D19 pi. Additionally, E2-137 treated mice showed significantly less severe disease on peak day (p<0.001) (FIG. 6).

At the peak of the disease on D19, eighteen EAE mice showed clinical disease score ≧2, however clinical disease score in 19 treated EAE mice was <1.5.

### Example 11

### A. Antibody Responses of E2-137-151 Peptide Treated and Untreated Mice with EAE, to E2-137 Peptide

Antibody Responses of RAE and E2 137-151 treated EAE mice to E2-137 and control peptide (E2 115-129) were measured by ELISA at the end of experiment on D39 pi. ELISA results showed that the antibody response of treated EAE mice to E2-137 was significantly higher than untreated EAE mice (p<0.01) on D39 pi. There was no significant difference between antibody responses of EAE and treated LAB to MOG 35-55 and control peptide (FIG. 7).

### Example 12

### B. Inflammation, Demyelination and Remyelination in E2-137-151 Peptide Treated and Untreated Mice with EAE

In order to be able to analyze de- and remyelination accurately in untreated and E2 137-151 treated LAE mice, one micron sections of lumbar spinal cords of all mice, were analyzed as described above for SFV-infected mice, on different days post immunization. Extensive inflammatory response and groups of demyelinated axons were seen in the white matter of the lumbar regions of spinal cords of untreated EAE mice (FIG. 8A, see arrows). Widespread Wallerian degeneration evidenced by dilated and collapsed myelin sheaths, some containing axonal remnants, was also observed. Cellular infiltration was present within the meningeal space as well. In contrast, the lumbar spinal cords of E2 137-151 treated, EAE mice, on day 33 pi, displayed little to no inflammation. Unlike untreated, sections from treated EAE mice exhibited some remyelinating fibers (FIG. 8B, see arrows). Spinal cord white matter of normal control mice is also shown for comparison (FIG. 8C)

### Example 13

### Direct Transfer of Antibodies against E2 137-151 to Mice with EAE

### Procedure

A first group of mice will receive 0.1 mg purified anti E2 137-151 antibody intraperitoneally by 4-8 injections from day 10-12 (At the onset of EAE disease) administered weekly or at 4-day intervals, for a total of 0.8 mg of antibody, over 32 days. The second group of mice will receive normal antibody, administered similarly in injections of 0.1 mg each for a total of 0.8 mg of antibody, during 32 days. Mice would be sacrificed on Day 45 and in all groups spinal cord demyelination and remyelination would be measured after sacrifice. The third group of mice will receive 1.2 mg antibody against E2 115-137, administered in 4 injections with weekly over 32 days. PBS will be administered to a control group of mice.

### Example 14

### Role of MBP Peptide-Specific Antibodies in Remyelination

Above studies indicated that production of antibody to SFV's epitope peptide, E2 137-151 and remyelination are the only two components that are both subnormal and deficient in KO mice. Therefore, we anticipate that remyelination is mediated through antibody production to E2 137-151. Anti SFV antibody also cross-reacts with the MBP peptide (56-68), which has mimicry with E2 137-151. Antibody responses of γδ-KO to mouse MBP epitope, 56-68 is also lower in KO than in WT mice.

It is also possible that remyelination occurs through the production of antibody to mouse MBP 56-68. As discussed in the Background of the Invention, anti MBP antibody has been associated with remyelination, in other systems.

Since E2 137-151 also has molecular mimicry with human MBP 102-118, immunization with former peptide can lead to production of antibodies to the latter peptide and remyelination in MS patients.

### Discussion

The Semliki Forest Virus (SFV) is one of the CNS viral models that have been used to study the pathogenesis of demyelinating diseases such as multiple sclerosis (1). Previous studies of TCR γδ cells in viral infections, EAE (39) and MS (38) have indicated that they may play a role in the recovery and repair of the CNS damage. Indeed, γδ⁺ T-cells were shown to accumulate and respond preferentially to heat shock protein Asp) expressed in damaged brain in MS (38). In this study, we have mainly found a role for γδ T-cells in antibody response and remyelination during SFV-infection.

SFV-infection causes paralysis in mice and leads to myelin injury after clearance of virus, followed by remyelination (6). Clinically, the KO mice showed the signs of illness earlier than WT mice and displayed a higher percentage of sick mice at the time of peak viral disease and inflammation, than the WT mice did. SFV-infected KO mice also did not clear the virus as well as the WT mice did, but it only took a few more days to do so. In summary, γδ T-cells appeared to play a significant role in clearance of SFV from the infected mice, however, in their absence; other mechanisms such as TCR αβ T cells were able to eventually clear the virus. In agreement with our report, vaccinia virus-infection of TCRδ-deficient mice resulted in a higher viral titers than normal mice (45). A study of influenza virus, has further indicated that, though necessary for viral clearance, γδ T cells alone were not sufficient for reduction/elimination of the viral load (48), as TCR αβ deficient mice were unable to clear virus even at time points well beyond the elimination of infectious virus by normal mice. Therefore, there appears to be an accessory role for TCR γδ cells in influenza virus infection by the production of cytokines that enhance the immune response.

In a typical SFV-infection of WT B6 mice, inflammation peaks on day 8-9 pi, greatly subsides after viral clearance, and is completely resolved after d21 pi. In SFV-infected KO mice, however, inflammation was not significantly stronger than in WT mice. Although γδ T-cells also play a role in EAE, the autoimmune inflammatory demyelinating model of MS, conflicting results have been reported. While some studies have found a potentiating effect of 78 T-cells on the severity of EAE (42) (43), others have suggested a suppressive effect (39, 40). Although the reason for this discrepancy (pathological γδ T-cells vs. protective γδ T-cells) is not clear at this time, results obtained in the collagen-induced arthritis model suggest both the inflammation-promoting and the suppressing role of TCR γδ⁺ T-cells, depending on the period in the disease process (74). FACS studies showed that CD3⁺CD45R⁻γδ⁺ T-cells of brain, from SFV-infected WT mice, peaked at the time of viral clearance and antibody response, on day 7 pi. γδ⁺ KO mice lacked TCR γδ T cells in the brains, spleen and IEL. These data further confirmed our conclusion that γδ⁺ T-cells contributed to viral clearance and resolution of inflammation. FACS analysis of BMNC from EAE mice (40) have shown that the percentage of CD3⁺TCR γδ⁺ cells significantly increased during the height of the acute phase, and again at the relapse of EAE, indicating their role in the pathogenesis of this autoimmune disease and further confirming the possibility of different roles that these cells can play in CNS pathology. Our FACS studies, however, indicated that unlike in SFV-infection, in acute EAE, TCRγδ⁺ T cells were fewer initially during the acute inflammation and increased later, both in intensity and proportion, during chronic stage. This may suggest their involvement in the recovery period (remittance) of EAE.

In SFV-infected brains, vacuolation is the typical pathology, seen to some extent on earlier days together with inflammation and destruction in the gray and white matter (2). Because of the limitations associated with using Luxol Fast Blue staining of paraffin-embedded sections, demyelination in the myelinated strip of cerebellum, seen later from day 15 pi on, is seen as vacuolation. In such sections, demyelination (vacuolation in the myelinated regions of cerebellum), was not significantly different in the WT and γδ KO mice. However, our previous studies using thin sections (5) have shown scattered fibers with primary demyelination in these myelinated areas of cerebellum and other CNS regions. The areas of “primary demyelination”, seen in white matter of SFV-infected WT B6 mice, on days 14-21 pi, quickly mix with thinly remyelinated fibers (5). Brains of SFV-infected KO mice, however, unlike the WT, were not remyelinated by day 35 pi. Although one may contribute this difference to a delayed remyelination due to somewhat more sustained inflammation in the brains of SFV-infected KO mice, the difference in inflammatory responses of these mice was minimal on days 15-35 pi (when remyelination takes effect) and cannot account for the difference in remyelination. The latter difference was clearly visible in three separate experiments and was attributed to the lack of γδ T cells.

Our previous studies have also shown that the late onset demyelination following SFV-infection (6) may have been in part mediated by antibodies (4). These antibodies were directed against SFV epitope E2115-131 which also reacted with its molecularly mimicked myelin peptide, MOG 18-32; since immunization of mice with this MOG peptide also induced demyelination, with similar histopathological features as those in SFV-infected mice (9). In other viral models, such as Mouse hepatitis virus (MHV)-induced demyelination, in nude mice, it was thought to be mediated by γδ⁺ T-cells, which substituted for the usual αβ⁺ T-cells, in this process (30). The effecter mechanism of γδT cellsin this process, however, has not been studied. As previously reported (4), mice inoculated with SFV develop antibodies to SFV and to E2 peptides, E2 137-151 and -E2 115-129. In this study, the reactivity of WT sera with the E2 137-151 began earlier and was significantly higher than the KO sera. The reactivity of sera from SFV-infected WT and K0 to E2 115-129 and to control peptide, however, was not significantly different. The titer of anti-E2 137-151 is always higher than the titer of antibodies anti-E2 115-129 antibodies (4). If antibodies against E2 137-151 were neutralizing antibodies (8), then it can be postulated that γδ T cells are involved in viral clearance by helping B cells produce antibodies against this T-dependent antigen (8). In VSV-infection, it was found that TCR γδ cells from TCR β-deficient mice were able to mount neutralizing IgG antibodies, albeit at lower levels than in normal mice (47). Although the mechanisms underlying this phenomenon is not clear; it appears that TCRγ cells are driving cognate interactions and subsequent isotype switching of anti-VSV-G protein specific IgM-expressing B cells, through a VSV-G protein-dependent mechanism.

Although γδ⁺ T-cells respond to lipids and non-protein antigens (32, 33), to our surprise, reactivity to non-protein cell surface component, Gal-C, and to rMOG, were equally high in both, SFV-infected WT and KO mice. Given that antibodies to myelin-specific glycolipid, Gal-C, and to MOG, both activate the same signaling pathway leading to myelin degradation, it has been proposed that there is a direct interaction between the membrane-associated regions of MOG and Gal-C (62) (75). Therefore, antibodies directed against Gal-C portion of SFV membrane could have led to disruption of MOG and induction of anti MOG antibodies, and to demyelination, in this model. Previous studies (76) have shown that VγVδ-TCR triggering resulted in prominent expression of essential B-cell costimulatory molecules, and provide potent B-cell help during in vitro antibody production. Collectively, our findings agree with a role for γδ T cells in humoral immunity during antiviral responses.

The most striking differences in the SFV-induced pathogenesis in WT and KO mice were in the antibody production against E2 137-151, and as discussed above, in remyelination, which were both significantly reduced in KO mice, as compared to WT mice. Previously, we have shown that in SFV-infected B-cell KO mice, where like TCR-γδ KO mice, viral clearance was delayed; numbers of inflammatory foci and PAS positive macrophages were not significantly different than WT mice. However, unlike SFV-infected γδ KO, the B-cell KO mice showed significantly fewer number of vaccuolation (demyelination) in the white matter. This indicated that it is the immune response to the virus and not virus itself that causes demyelination; the point which was originally shown by (2) using SFV-infection of nude mice. Consequently, in γδ KO mice, the absence of complete remyelination could not be explained by simple delay in the whole process of viral clearance followed by de- and remyelination. The lack of γδ T cells, however, appeared to only affect antibody production to one of the envelope-associated peptide, E2 137-151(8). Histologically, SFV-infected γδ KO mice did not properly remyelinate after viral clearance, as the SFV-infected WT mice did, by day 35 pi. Since demyelination is not due to presence of viral replication, the delay in viral clearance could not have resulted in delay in remyelination. Previously, it has been clearly shown that in the absence of T cells (nude mice) (3) or B cells (4), which both result in the persistence of viral replication in the CNS tissue, no demyelination occurred. The absence of complete remyelination, however, had a strong correlation with the low amount of anti E2 137-151 antibodies, and could be attributed to the cause of it.

We have previously found significant homology between the amino acid sequence of E2 137-151 peptide and MBP peptides, 54-68 and 64-75 (9). These mimicked peptides also demonstrated cross recognition by antibody response.

Our studies also indicated that the treatment of EAE mice with E2 137-151 peptide significantly suppressed the clinical disease (p<0.001). This may be due to the production of antibodies which cross reacted with MBP mimicked peptides. Indeed, we have also shown in preliminary experiments that the sera from E2 137 treated EAE mice also reacted with MBP 54-68. Furthermore, it appeared that the spinal cords of E2 137 treated EAE mice were remyelinating during this treatment.

It has been shown that the recombinant form of an IgM autoantibody, rHIgM22, prepared from sera of individuals with a form of monoclonal gammopathy has remyelinating capabilities, when tested in Theiler's virus model (56-60). This autoantibody was shown to mediate the rescue of an oligodendrocyte cell line, CG4 cells, in a Ca²⁺ influx and lipid raft motility dependent way by the suppression of caspase-3 activation and apoptosis (66). Thus, it is possible that the binding of certain autoantibodies to oligodendrocytes and subsequent influx of Ca²⁺ play a part in the regulation of oligodendrocyte structure, function, rescue from apoptosis and remyelination. Previous studies have also indicated that anti MBP antibody production induces suppression of EAE, remyelination (61, 63) and promotes recovery. Using similar mechanisms as above, antibodies produced against the envelope-associated E2 peptide 137-151 of SFV, which also has molecular mimicry with a MBP peptide, may play a part in remyelination, after SFV-induced immune-mediated demyelination. The anti E2 137-151 antibodies may take part in this mechanism by binding to SFV-infected oligodendrocytes, through which these cells may survive and induce a rapid growth and repair of SFV-induced damage to myelin. In this model, the helper cells in the production of such antibodies may be γδ T cells. It has been found that treatment with remyelinating antibodies, unlike anti T cell and anti MQ antibody treatments, will not be immunosuppressive.

These studies have also shown that immunization with E2 137-151 peptide increased the antibody to this peptide only and promoted remyelination in KO mice.

Clinically, signs of encephalomyelitis in SFV-infected KO mice, treated with this peptide were significantly suppressed. Furthermore, the immunization with E2 137-151 peptide also improved clinical EAE and induced remyelination in these mice. Elucidation of treatment protocols by which this remyelination-promoting antibody exert its beneficial effect is worthwhile in the overall development of targeted therapies for MS, especially with regard to heterogeneity in the etiologies of demyelination and patterns of remyelination in different forms of MS (69).

### REFERENCES

1. Fazakerley, J. K., Amor, S., Nash, A. A. Animal Model Systems of MS. Molecular Biology of Multiple Sclerosis. Russell, W. C. (Ed.), C. Wiley, Chichester. 1997, 255-273
2. Fazakerley J K (2002) Pathogenesis of Semliki Forest virus encephalitis. J Neurovirol 8 Suppl 2: 66-74.
3. Subak-Sharpe I, Dyson H, Fazakerley J (1993) In vivo depletion of CD8+ T cells prevents lesions of demyelination in Semliki Forest virus infection. J Virol 67: 7629-7633.
4. Smith-Norowitz T A, Sobel R A, Mokhtarian F (2000) B cells and antibodies in the pathogenesis of myelin injury in Semliki Forest Virus encephalomyelitis. Cell Immunol 200: 27-35.
5. Mokhtarian, F., Huan, C., Roman, C., Raine, C. S. (2003) Semliki Forest Virus-induced demyelination and remyelination-involvement of B-cells and anti-myelin antibodies. J. Neuroimmunol. 137: 19-31
6. Mokhtarian F, Swoveland P (1987) Predisposition to EAE induction in resistant mice by prior infection with Semliki Forest virus. I Immunol 138: 3264-3268.
7. Boere W A, Benaissa-Trouw B J, Harmsen M, Kraaijeveld C A, Snippe H (1983) Neutralizing and non-neutralizing monoclonal antibodies to the E2 glycoprotein of Semliki Forest virus can protect mice from lethal encephalitis. J Gen Virol 64 (Pt 6): 1405-1408.
8. Snijders A, Benaissa-Trouw B J, Oosterlaken T A, Puijk W C, Posthumus W P, Meloen R H, Boere W A, Oosting J D, Kraaijeveld C A, Snippe H (1991) Identification of linear epitopes on Semliki Forest virus E2 membrane protein and their effectiveness as a synthetic peptide vaccine. J Gen Virol 72 (Pt 3): 557-565.
9. Mokhtarian F, Zhang Z, Shi Y, Gonzales E, Sobel RA (1999) Molecular mimicry between a viral peptide and a myelin oligodendrocyte glycoprotein peptide induces autoimmune demyelinating disease in mice J Neuroimmunol 95: 43-54.
10. Gold R, Linington C, Lassman H. (2006) Understanding pathogenesis and therapy of multiple sclerosis via animal models: 70 years of merits and culprits in experimental autoimmune encephalomyelitis research. Brain. 129.1953-71. Review.
11. Pender M P, Greer J M (2007). Immunology of multiple sclerosis. Curr Allergy Asthma Rep. 7(4):285-92. Review.
12. Sobel, R A., Creer, J. M. and Kuchroo, V. K. (1994). Minireview: autoimmune responses to myelin proteolipid protein. Neurochem Res. 19:915-21.
13. Greer, J. M., Sobel, R. A., Sette, A., Southwood, S., Lees, M. B. And Kuchroo, V. K. (1996). Immunogenic and encephalitogenic epitope clusters of myelin proteolipid protein. J. Immunol. 156:371-379.
14. Whitham, R. H., Jones, R. E., Hashim, G. A, et al (1991). Location of a new encephalitogenic epitope in proteolipid protein that induces relapsing EAE in PL/J and (SJL×PL)F1 mice. J. Immunol. 147:3803-08.
15. Mendel, I., Kerlero de Rosbo, N. and Ben-Nun, A. (1995). A myelin oligodendrocyte glycoprotein peptide induces typical chronic EAE in H-2b mice: fine specificity and T cell receptor Vb expression of encephalitogeniic T cells. Eur. J. Immunol. 25:1951-1959.
16. Kerlero de Rosbo, N., Mendel, I. Ben-Nun, A. (1995). Chronic relapsing EAE with a delayed onset and an atypical clinical course, induced in PL/J mice by (MOG)-derived peptide: preliminary analysis of MOG T cell epitopes. Eur. J. Immunol. 25:985-993.
17. Amor, S., Groome, N., Linington, C., Morris, M. M., Dommair, K., Gardinier, M. V., Matthieu, J. M. and Baker, D. (1994). Identification of epitopes of myelin oligodendrocyte glycoprotein for the induction of experimental allergic encephalomyelitis in SJL and Biozzi AB/H mice. J. Immunol. 153:4349-4356.
18. Johns, T. G., Kerlero de Rosbo, N., Menon, K. K., Abo, S., Gonzales, M. F. and Bernard, C. C. A. (1995). Myelin oligodendrocyte glycoprotein induces a demyelinating encephalomyelitis resembling multiple sclerosis. J. Immunol. 154:5536-5541.
19. Tuohy, V. K., Sobel, R. A., and Lees, B. (1988). Myelin proteolipid protein-induced experimental allergic encephalomyelitis: variations of disease expression in different strains of mice. J. Immunol. 140:1868-1873.
20. Mokhtarian, F., McFarlin, D. E. and Raine. C. S. (1984). Adoptive transfer of myelin basic protein sensitized T cells produces chronic relapsing demyelinating disease in mice. Nature 309:356-358.
21. Raine, C. S., F. Mokhtarian, and McFarlin. D. E. (1984). Adoptively transferred chronic relapsing experimental autoimmune encephalomyelitis in the mouse. Neuropathologic analysis. Lab. Invest. 51:534-546.
22. Beraud, E., Reshef, T., Vandenbark, A. A., Offner, H., Fritz, R., Chou., C.-H., Bernard, D. and Cohen. I. R. (1986). Experimental autoimmune encephalomyelitis mediated by T lymphocyte lines: genotype of antigen-presenting cells influences immunodominant epitope of basic protein. J. Immunol. 136:511-517.
23. Zamvil, S. S., Mitchell, D. J., Moore, C. A., Kitamura, K., Steinman, L. and Rothbard, J. B. (1986). T-cell epitope of the autoantigen myelin basic protein that induces encephalomyelitis. Nature 324:258-260.
24. Mokhtarian F (1988). Role of Ia antigen in the induction of adoptively transferred acute and chronic relapsing demyelinating disease in mice. Clin. Immunol. Immunopath. 49:308-317
25. Mokhtarian F. T. Shirazian. O. Batuman and Y Shi (1996) The effects of oral myelin basic protein and Dexamethasone treatment on EAE. In: Oral toleranse: mechanisms and applications Ann. N.Y. Acad. Sci. 778:414-418.
26. Moses Rodriguez, David J. Miller, Vanda A. Lennon; Immunoglobulins reactive with myelin basic protein promote CNS remyelination, NEUROLOGY 1996; 46:538-545
27. Lanier L L, Federspiel N A, Ruitenberg J J, Phillips J H, Allison J P, Littman D, Weiss A (1987) The T cell antigen receptor complex expressed on normal peripheral blood CD4-, CD8-T lymphocytes. A CD3-associated disulfide-linked gamma chain heterodimer. J Exp Med 165: 1076-1094.
28. Szymanska B, Rajan A J, Gao Y L, Tronczynska E, Brosnan C F, Selmaj K (1999) Evidence for gammadelta T cells with a restricted Vgamma6 junctional region in the normal mouse central nervous system. J Neuroimmunol 100: 260-265.
29. Zheng B, Marinova F, Han J, Tan T H, Han S (2003) Cutting edge: gamma delta T cells provide help to B cells with altered clonotypes and are capable of inducing Ig gene hypermutation. J Immunol 171: 4979-4983.
30. Dandekar A A, Perlman S (2002a) Virus-induced demyelination in nude mice is mediated by gamma delta T cells. Am J Pathol 161: 1255-1263.
31. Schild H, Mavaddat N, Litzenberger C, Ehrlich E W, Davis M M, Bluestone J A, Matis L, Draper R K, Chien Y-H. (1994) The nature of major histocompatibility complex recognition by γδ T-cells. Cell 76: 29-37.
32. Tanaka Y, Morita C T, Tanaka Y, Nieves B, Brenner M B, Bloom B R (1995) Natural and synthetic non-peptide antigens recognized by human gamma delta T cells. Nature 375: 155-158.
33. Tanaka Y, Sano S, Nieves E, De Libero G, Rosa D, Modlin R L, Brenner M B, Bloom B R, Morita C T (1994) Nonpeptide ligands for human gamma delta T cells. Proc Natl Acad Sci USA 91: 8175-8179.
34. Porcelli, S, Morita, C, Modlin, R. (1996) T cell recognition of non-peptide antigens. Cur. Opin. Immunol. 8: 510-516.
35. Constant P, Davodeau F, Peyrat M A, Poquet Y, Puzo G, Bonneville M, et al. (1994) Stimulation of human gamma delta T cells by nonpeptidic mycobacterial ligands. Science. 264: 267-270.
36. Sim, G.-K, Olsson, C., Augustin, A. (1995) Commitment and Maintenance of the αβ and γδ T Cell Lineages. J. Immunol. 154: 5821-5831.
37. Hayday A, Tigelaar R (2003) Immunoregulation in the tissues by gammadelta T cells. Nat Rev Immunol 3: 233-242.
38. Selmaj K, Brosnan C F, Raine C S (1991) Colocalization of lymphocytes bearing gamma delta T-cell receptor and heat shock protein hsp65+ oligodendrocytes in multiple sclerosis. Proc Natl Acad Sci USA 88: 6452-6456.
39. Kobayashi Y, Kawai K, Ito K, Honda H, Sobue G, Yoshikai Y (1997) Aggravation of murine experimental allergic encephalomyelitis by administration of T-cell receptor gammadelta-specific antibody. J Neuroimmunol 73: 169-174
40. Rajan A J, Klein J D, Brosnon C F. The effect of gammadelta T cell depletion on cytokine gene expression in experimental allergic encephalomyelitis. J. Immunol. 1998; 160(12):5955-62.
41. Rajan A J, Asensio V C, Campbell I L, Brosnon C F. Experimental autoimmune encephalomyelitis on the SJL mouse: effect of gamma delta T cell depletion on chemokine and chemokine receptor expression in the central nervous system. J. Immunol 2000; 164(4):2120-30.
42. Ponomarev E D, Novikova M, Yassai M, Szczepanik M, Gorski J, Dittel B N (2004) Gamma delta T cell regulation of IFN-gamma production by central nervous system-infiltrating encephalitogenic T cells: correlation with recovery from experimental autoimmune encephalomyelitis. J Immunol 173: 1587-1595.
43. Ponomarev E D, Dittel B N (2005) Gamma delta T cells regulate the extent and duration of inflammation in the central nervous system by a Fas ligand-dependent mechanism. J Immunol 174: 4678-4687.
44. Sciammas R, Johnson R M, Sperling A I, Brady W, Linsley P S, Spear P G, Fitch F W, Bluestone J A (1994) Unique antigen recognition by a herpesvirus-specific TCR-gamma delta cell. J Immunol 152: 5392-5397.
45. Selin L K, Santolucito P A, Pinto A K, Szomolanyi-Tsuda E, Welsh R M (2001) Innate immunity to viruses: control of vaccinia virus infection by gamma delta T cells. J Immunol 166: 6784-6794.
46. Huber S A, Graveline D, Born W K, O'Brien R L (2001) Cytokine production by Vgamma(+)-T-cell subsets is an important factor determining CD4(+)-Th-cell phenotype and susceptibility of BALB/c mice to coxsackievirus B3-induced myocarditis. J Virol 75: 5860-5869.
47. Maloy K J, Odermatt B, Hengartner H, Zinkemagel R M (1998) Interferon gamma-producing gammadelta T cell-dependent antibody isotype switching in the absence of germinal center formation during virus infection. Proc Natl Acad Sci USA 95: 1160-1165.
48. Carding S R, Allan W, Kyes S, Hayday A, Bottomly K, Doherty P C (1990) Late dominance of the inflammatory process in murine influenza by gamma/delta+T cells. J Exp Med 172: 1225-1231.
49. Fujinami, R. S. and M. B. A. Oldstone. (1985) Amino acid homology between the encephalitogenic site of myelin basic protein and virus: Mechanism for autoimmunity. Science 230:1043-1045.
50. Waksman, B., Mechanisms in multiple sclerosis. (1985) Nature 318: 104-105.
51. Ercolini A M, Miller S D. Mechanisms of immunopathology in murine models of central nervous system demyelinating disease J. Immunol. 2006 Mar. 15; 176(6):3293-8. Review.
52. Ercolini A M, Miller S D. Role of immunologic cross-reactivity in neurological diseases. Neurol Res. 2005 October; 27(7):726-33. Review.
53. Miyazaki, I., R. K. Cheung, R. Gaedigk, M. F. Hui, J. Van der Meulen, R. V. Rajotte, and H. M. Dosch. (1995) T cell activation and anergy to islet cell antigen in type I diabetes. J. Immunol. 154: 1461-1469.
54. Mamula, M. J., S. Fatenejad, and J. Craft. (1994) B cells process and present lupus autoantigens that initiate autoimmune T cell responses. J. Immunol. 152: 1453-1461.
55. C. S. Reiss, D. A. Chesler, J Hodges, D. D. C. Ireland and N. Chen, Innate immune responses in viral encephalitis. In: B. Dietzscholdand J. A. Richt, Editors, Protective and Pathological Immune Responses in the CNS, Current Topics in Microbiology and Immunology vol. 265, Springer (2002), pp. 63-94
56. Miller, D. J., Asakura, K., Rodriguez, M. (1995) Experimental Strategies to Promote Central Nervous System Remyelination in Multiple Sclerosis: Insights Gained from the Theiler's Virus Model System. J. Neurosci Res. 41: 291-296.
57. Miller, D. J., Sanhorn, K. S., Katzman, J. A., Rodriguez, M. (1994) Monclonal Auto-antibodies Promote Central Nervous System Repair in an Animal Model of Multiple Sclerosis. J. Neurosci. 14: 6230-6238
58. Miller, D. J., Bright, J. J., Sriram, S., Rodriguez, M. (1997) Successful Treatment of Established Relapsing Experimental Autoimmune Encephalomyelitis in Mice with a Monoclonal Natural Antibody. J. Neuroimmunol. 75: 204-209.
59. Pavelko, K. D., van Englelen, B. G., Rodriguez, M. (1998) Acceleration in the Rate of CNS Remyelination in Lysolecithin-induced Demyelination. J. Neurosci. 18: 2498-2505.
60. Warrington, A. E., Asakura, K., Biebert, A. J., Ciric, B., VanKeulen, V., Kaveri, S, V., Kyle, R, A., Pease, L. R., Rodriguez, M. (2000) Human Monoclonal Antibodies Reactive to Oligodendrocytes Promote Remyelination in a Model of Multiple Sclerosis. Proc. Natl. Acad. Sci. USA 97(12): 6820-6825
61. Moses Rodriguez, David J. Miller, Vanda A. Lennon ((1996); Immunoglobulins reactive with myelin basic protein promote CNS remyelination, NEUROLOGY 1996; 46:538-545
62. Kroepfl J F, Vise L R, Charron A J, Linington C, Gardinier M V (1996) Investigation of myelin/oligodendrocyte glycoprotein membrane topology. J Neurochem 67: 2219-2222.
63. Mateo Paz Soldan, M., Rodriguez, M. (2002) Heterogeneity of Pathogenesis in Multiple Sclerosis: Implications for Promotions of Remyelination. J. Infect. Dis. 186(2): S248-S253.
64. Bogoljub Cirica, Virginia Van Keulena, Mateo Paz Soldan, Moses Rodriguez, Larry R. Pease (2004). Antibody-mediated remyelination operates through mechanism independent of immunomodulation. J Neuroimmunol, 146:153-161
65. Rodriguez, Moses, Effectors of Demyelinationi and Remyelination in the CNS: Implications for Multiple Sclerosis. Brain Pathology, 17:219-229 (2007)
66. Howe, C. L., Bieber, A. J., Warrington, A. E., Pease, L. R., Rodriguez, M. (2004) Antiapoptotic Signaling by a Remyelination-Promoting Human Antimyelin Antibody. Neurobiol. Dis. 15: 120-131.
67. Teitlebaum D, Aharoni, R, Sela, M, and Arnon, R (1991) Crossreactions and specificities of monoclonal antibodies against myelin basic protein and against the synthetic copolymer-1 Proc. Natl. Acad. Sci. USA 88 9525-9532.
68. Daren R. Ure* and Moses Rodriguez (2002). Polyreactive antibodies to glatiramer acetate promote myelin repair in murine model of demyelinating disease. The FASEB J. express article 10.1096/fj.01-1023′fje. Published online Jun. 7, 2002.
69. Peter Patrikios, Christine Stadelmann, Alexandra Kutzelnigg, Helmut Rauschka, Manfred Schmidbauer, Henning Laursen, Per Soelberg Sorensen, Wolfgang Brück, Claudia Lucchinetti and Hans Lassmann. Remyelination is extensive in a subset of multiple sclerosis patients, Brain 2006 129(12): 3165-3172.
70. Itohara S, Mombaerts P, Lafaille J, Iacomini J, Nelson A, Clarke A R, Hooper M L, Farr A, Tonegawa S (1993) T cell receptor delta gene mutant mice: independent generation of alpha beta T cells and programmed rearrangements of gamma delta TCR genes. Cell 72: 337-348.
71. McGavern, D B, Murray, P D. and Rodriguez, M (1999) Quantitation of Spinal Cord Demyelination, Remyelination, Atrophy, and Axonal Loss in a Model of Progressive Neurologic Injury J. Neurosci Res, 58:492-504.
72. Fernandez I M, Harmsen M, Benaissa-Trouw B J, Stuij I, Puyk W, Meloen R H, Snippe H, Kraaijeveld C A. Epitope polarity and adjuvants influence the fine specificity of the humoral response against Semliki Forest virus specific peptide vaccines. Vaccine. 1998 October; 16(16):1531-6.
73. Hassen, G W, J. Feliberti, L. Kesner, A. Stracher and F. Mokhtarian, (2006). The role of a Novel Protease Inhibitor in acute EAE. J. Neuroimmunol. 180; 135-146, 2006.
74. Peterman G M, Spencer C, Sperling A I, Bluestone J A (1993) Role of gamma delta T cells in murine collagen-induced arthritis. J Immunol 151: 6546-6558.
75. Johns T G, Bernard C C (1999) The structure and function of myelin oligodendrocyte glycoprotein. J Neurochem 72: 1-9.
76. Brandes M, Willimann K, Lang A B, Nam K H, Jin C, Brenner M B, Morita C T, Moser B (2003) Flexible migration program regulates gamma delta T-cell involvement in humoral immunity. Blood 102: 3693-3701.

TABLE I
Clinical Stages of SFV-Infection in B6 and γδ KO mice

| | | | | Number of mice^{a} at | | | | | | |
|---|---|---|---|---|---|---|---|---|---|---|
| | Day after | No. | No. | stages of infection^{b} | | | | | | |
| Mice | Infection | mice | sacrificed | 0 | 1 | 2 | 3 | 4 | 5 | 6 |
| B6 | 0 | 81 | 15 | 79 | 0 | 0 | 0 | 0 | 0 | 0 |
| | 4 | 66 | 9 | 64 | 0 | 0 | 0 | 0 | 0 | 0 |
| | 7* | 57 | 18 | 23 | 8 | 8 | 10 | 4 | 1 | 3 |
| | 10 | 39 | 9 | 15 | 7 | 7 | 8 | 2 | 0 | 0 |
| | 14 | 30 | 9 | 14 | 5 | 5 | 6 | 0 | 0 | 0 |
| | 21 | 21 | 12 | 12 | 3 | 2 | 4 | 0 | 0 | 0 |
| | 35 | 9 | 9 | 9 | 0 | 0 | 0 | 0 | 0 | 0 |
| KO | 0 | 57 | 9 | 57 | 0 | 0 | 0 | 0 | 0 | 0 |
| | 4 | 48 | 6 | 45 | 2 | 0 | 0 | 1 | 0 | 0 |
| | 7* | 42 | 9 | 13 | 2 | 2 | 15 | 3 | 4 | 3 |
| | 10 | 33 | 6 | 13 | 2 | 1 | 7 | 2 | 2 | 0 |
| | 14 | 27 | 9 | 13 | 2 | 0 | 2 | 0 | 1 | 0 |
| | 21 | 18 | 9 | 9 | 0 | 0 | 0 | 0 | 0 | 0 |
| | 35 | 9 | 9 | 9 | 0 | 0 | 0 | 0 | 0 | 0 |

^{a}The discrepancies in number of mice at each stage on different days pi, are due to the deduction of number of mice sacrificed for virological, flow cytometric and histological analysis, all of which had significant clinical symptoms. Data pooled from 3 separate experiments.
^{b}Stages 1-3 represent temporary general weakness attributable mainly to effects of viral infection. Stages 4-6 are neurological signs, i.e. mild to severe paralysis, attributable to immunopathology (see results).
*Average Clinical Scores of KO (2.4) > WT (1.6), p < 0.01, as determined by a two-tailed student t test.

TABLE II
Inflammation in the Brains of SFV-infected WT and γδKO mice
on Days Postinfection^{a}

| | |
|---|---|
| Day | Average Number of Foci |

| post-infection | WT | KO |
|---|---|---|
| 0 | 0, 0, 0 (0.00) | 0, 0, 0 (0.00) |
| 7 | 25, 16, 21 (20.6) | 23, 19, 30 (24.0) |
| 14 | 4, 7, 8 (6.3) | 3, 10, 12 (8.3) |
| 21 | 3, 3, 6 (4.0) | 3, 6, 5 (4.7) |
| 35 | 1, 0, 1 (0.6) | 0, 1, 2 (1.0) |
| 42 | 0, 0, 0 (0.0) | 0, 0, 0 (0.0) |

^{a}Inflammation scoring is described in Materials and Methods. Each number is an average of the total number of inflammatory foci in one brain, using H&E stain. Three separate experiments were performed.

TABLE III
Demyelination in the Brains of SFV-infected WT and γδ KO mice
on Days Postinfection^{a}

white matter
vacuolation scores (avg.)

| Day post-infection | WT | KO |
|---|---|---|
| 0 | 0, 0, 0 (0.00) | 0, 0, 0 (0.0) |
| 7 | 1, 1, 1.5 (1.17) | 1, 1.5, 2 (1.5) |
| 14 | 1.5, 2 (1.75) | 1.5, 2. 1 (1.5) |
| 21 | 3, 1.5, 2.5 (2.3) | 1.5, 2, 3 (2.17) |
| 35* | 0.5, 0.5, 0.5 (0.5) | 2, 1.5, 1.5 (1.6) |

^{a}Demyelination scoring is described in Materials and Methods. Each number is an average of the total number of vacuolation in one brain, using LFB stain. Three separate experiments were performed.
*Average vacuolation Score, KO > WT, p < 0.005; determined by two-tailed student t test.

TABLE IV
Viral titration of SFV-infected WT and KO mice
On Days Post infection

| | Viral titers (−log₁₀ TCID₅₀ units | | | | | | |
|---|---|---|---|---|---|---|---|
| | in 0.1 ml) on days pi | | | | | | |
| Mice | Day 0 | Day 3 | Day 5 | Day 7 | Day 10 | Day 14 | Day 21 |
| WT | 0.00 | 2.75 | 4.00 | *N | N | N | N |
| KO | 0.00 | 3.00 | 4.00 | 3.75 | 2.00 | N | N |

Brain samples were obtained from a pool of 2-3 C57BL/6 (WT) and B6.129P2-Tcrd^{tm1Mom} (KO), at each indicated time point, post SFV-infection, from three experiments. TCID₅₀ assay was carried out as described in Materials and Methods.
*N = Negative.
*p < 0.01, WT > KO, on day 21 and p < 0.05, W > KO, on day 35.

TABLE V
Peptide Alignments of E2 and Myelin Proteins

| | | | | |
|---|---|---|---|---|
| Identity | Peptides | Sequence* | # AA | % |

| | | | | |
|---|---|---|---|---|
| A) | E2 118-129 | TRNAVRACRIQY (SEQ ID NO: 42) | 12 | 33 |
| | & | :...::: | | |
| | MOG 18-29 | GDEAELPCRISP (SEQ ID NO: 43) | | |
| B) | E2 137-149 | GREKFTIRPHYGK (SEQ ID NO: 44) | 13 | 33 |
| | & | :... :. :::. | | |
| | MBP 56-68 | GKDSHTRTTHYGS | | |
| C) | E2 118-129 | TRNAVRACRIQY (SEQ ID NO: 45) | 12 | 17 |
| | | :... .. ..: | | |
| | MBP 64-75 | THYGSLPQKSQR (SEQ ID NO: 46) | | |
| D) | E2 118-129 | TRNAVRACRIQY (SEQ ID NO: 47) | 12 | 9 |
| | | . ...:. | | |
| | MBP 54-65 | GSGKDSHTRTTH (SEQ ID NO: 48) | | |

* A double dot represents complete homology and a single dot indicates partial homology.
Note:
The peptides shown in the alignments are sometimes shorter than the synthesized peptide.

TABLE VI
The net increase in the levels of antibody in SFV-infected mice

| | Net change in the levels of antibody | | | | |
|---|---|---|---|---|---|
| | in post infection days (ΔOD) | | | | |
| Antigens | D0 | D7-9 | D14-16 | D21-23 | D31-35 |
| SFV | 0 | 1.84 ± 0.21 | 1.79 ± 0.22 | 1.89 ± 0.27 | 2.10 ± 0.46 |
| E2 1370 | 0.84 ± 0.39 | 1.83 ± 0.47 | 2.19 ± 0.76 | 2.08 ± 0.67 | |
| E2 1150 | 0.82 ± 0.40 | 0.91 ± 0.14 | 0.95 ± 0.22 | 0.85 ± 0.07 | |
| MOG18 | 0 | 0.34 ± 0.31 | 0.75 ± 0.23 | 0.91 ± 0.15 | 0.91 ± 0.36 |
| MOG 35 | 0 | 055 ± 0.08 | 0.78 ± 0.08 | 0.82 ± 0.05 | 0.78 ± 0.07 |
| MBP 56 | 0 | 1.10 ± 0.04 | 0.83 ± 0.10 | 1.07 ± 0.12 | 0.55 ± 0.18 |
| FB | 0 | 0.06 ± 0.07 | 0.05 ± 0.07 | 0.05 ± 0.08 | 0.08 ± 0.09 |
| BSA | 0 | 0.12 ± 0.09 | 0.18 ± 0.09 | 0.15 ± 0.08 | 0.03 ± 0.04 |
| MOG | 0.47 ± 0.21 | 0.84 ± 0.18 | 0.91 ± 0.11 | 0.96 ± 0.07 | |
| Protein 0 | | | | | |
| MBP | 0.53 ± 0.11 | 1.04 ± 0.24 | 1.12 ± 0.31 | 1.00 ± 0.32 | |
| Protein 0 | | | | | |

calculated by subtracting day 0 optical density from the other post-infection days' optical densities (D × OD D0OD = ΔOD).

TABLE VII

| | | | | |
|---|---|---|---|---|
| Antigens | Dh* / D0 | Dh−D0 | | % |
| Identity** | (Ratio) | (difference) | AA Sequence** | Identity |

| | | | | |
|---|---|---|---|---|
| SFV | 3.129 | 1.673 | | |
| E2 137-151 | 3.96 | 0.406 | GREKFTIRPHYGKEI | |
| E2 115-129 | 5.28 | 0.458 | IQDTRNAVRASRIQY (SEQ ID NO: 49) | |
| MBP 64-75 | 2.93 | 0.375 | THYGSLPQKSQR | 17 |
| MOG 18-32 | 3.2 | 0.919 | DEAELPSRISPGKNA (SEQ ID NO: 50) | 33 |
| MOG 35-55 | 5.11 | 0.362 | MEVGWYRSPFSRVVHLYRNGK | |
| MBP 56-68 | 5.37 | 0.824 | GKDSHTRTTHYGS | 33 |
| FB | 1.26 | 0.068 | EGVNDNEEGFFSAR (SEQ ID NO: 51) | |
| BSA | 1.8 | 0.189 | | |
| MOG Protein | 4.1 | 0.89 | | |
| MBP Protein | 2.42 | 0.967 | | |

**See Table I for details of molecular mimicry

TABLE VIII
Rabbit Anti-SFV polyclonal^{a} antibody response to various viral and
myelin antigens, OD at 490 nm.

| | Rabbit | | |
|---|---|---|---|
| Antigens | Anti-SFV Ab | Normal Rabbit Serum | OD Difference |
| UV-SFV | 1.053 | −0.004 | 1.057 |
| ^{b}FGF 106-120 | 0.397 | 0.070 | 0.327 |
| E2 115-131 | 1.372 | 0.171 | 1.201 |
| E2 137-151 | 1.107 | 0.173 | 0.934 |
| MOG 18-32 | 0.907 | 0.260 | 0.647 |
| MOG 35-55 | 0.184 | 0.007 | 0.177 |
| rMOG | 0.359 | 0.213 | 0.146 |
| Bovine MBP | 0.622 | 0.283 | 0.339 |
| MBP 56-68 | 0.852 | 0.195 | 0.691 |

^{a}Anti-SFV IgG from pooled sera of 3 rabbits immunized with UV-inactivated SFV (A774 strain) and Normal IgG from nonimmune rabbit sera were commercially prepared, as described in Methods. Indirect ELISA was performed, using 10⁻² diluted sera, against UV-SFV, E2 peptides, recombinant MOG, MOG peptides, bovine MBP, and FGF.
^{b}FGF is fibroblast growth factor. FGF peptide 106-120 is used as an unrelated peptide control antigen.

TABLE IX
The net change in the level of antibody (nano gr ± SD) in post infection days

| | Net change in the levels of | | | | | | | | | |
|---|---|---|---|---|---|---|---|---|---|---|
| | antibody in post infection days | | | | | | | | | |
| | D0 | | D7 | | D14 | | D21 | | D35 | |
| Antigen | WT | KO | WT | KO | WT | KO | WT | KO | WT | KO |
| SFV | 0 | 0 | 146 ± 19 | 77 ± 47 | 520 ± 20 | 365 ± 33 | 640 ± 22 | 381 ± 42 | 992 ± 17 | 602 ± 33 |
| E2-137 | 0 | 0 | 72 ± 28 | 39 ± 25 | 565 ± 33 | 36 ± 19 | 1196 ± 61 | 46.5 ± 19 | 951 ± 50 | 41 ± 28 |
| E2 115 | 0 | 0 | 69 ± 29 | 65 ± 35 | 83 ± 16 | 34 ± 24 | 90 ± 20 | 55 ± 20 | 73 ± 14 | 29 ± 22 |
| PPD | 0 | 0 | 20 ± 14 | 38 ± 14 | 30 ± 17 | 29 ± 13 | 32 ± 21 | 20 ± 14 | 29 ± 19 | 29 ± 17 |

What is claimed is:

1. A method for enhancing remyelination in the central nervous system (CNS) of a mammalian subject comprising administering to the subject an effective amount of Semliki Forest Virus (SFV) epitope E2 137-151 peptide or homolog thereof and a pharmaceutically acceptable carrier.

2. A method for enhancing remyelination in the CNS of a mammalian subject comprising administering to the subject an effective amount of anti-E2 137-151 peptide antibody or homolog thereof.

3. A method for treating a CNS disease manifesting the clinical characteristics associated with damaged myelin in a mammalian subject by administering to the subject a therapeutically effective amount of E2 137-151 peptide or homolog thereof and a pharmaceutically acceptable carrier.

4. A method for treating a CNS disease manifesting the clinical characteristics associated with damaged myelin in a mammalian subject by administering to the subject a therapeutically effective amount of an E2 137-151 antibody or homolog thereof.

5. The method of claim 3, wherein the CNS disease is multiple sclerosis (MS).

6. The method of claim 4, wherein the CNS disease is multiple sclerosis (MS).

7. The method of claim 1, wherein the E2 137-151 peptide or homolog thereof is in the form of a polymer of E2 137-151 peptide or homolog thereof.

8. The method of claim 3, wherein the E2 137-151 peptide or homolog thereof is in the form of a polymer of E2 137-151 peptide or homolog thereof.

9. The method of claim 1, further enhancing the production of γδ T-cell receptor (TCR γδ) T cells in the subject simultaneously with, or sequentially to, the administration of the E2 137-151 peptide.

10. The method of claim 1, wherein the E2 137-151 peptide or homolog thereof is administered subcutaneously.

11. The method of claim 1, wherein anti E2 137-151 antibody or homolog thereof is administered intravenously.

12. The method of claim 1, wherein the homolog of E2 137-151 peptide comprises the amino acid sequence HYG and is homologous to mouse MBP 56-68 peptide.

13. The method of claim 12, wherein the mouse MBP 56-68 peptide has the amino acid sequence GKDSHTRTTHYGS (SEQ ID NO: 2).

14. The method of claim 1, wherein the homolog of E2 137-15 peptide comprises the amino acid sequence GRE and is homologous to human MBP 102-118 peptide.

15. The method of claim 14, wherein the human MBP 102-118 peptide has the amino acid sequence GREDNTFKDRPSESDEL (SEQ ID NO: 3).

16. The method of claim 1, wherein the E2 137-151 peptide has the amino acid sequence GREKFTIRPHYGKEI (SEQ ID NO: 1).

17. A monoclonal anti E2-137-151 antibody or a monoclonal anti homologue antibody.

18. A humanized monoclonal anti E2-137-151 antibody or a humanized monoclonal anti homologue antibody.


SEQUENCE LISTING (49 sequences)

SEQ ID NO: 1 - protein, 15 aa, Artificial Sequence
  Semliki Forest Virus membrane associated epitope E2 137-151
GREKFTIRPHYGKEI

SEQ ID NO: 2 - protein, 13 aa, Artificial Sequence
  Mouse Myelin Basic Protein (56-68)
GKDSHTRTTHYGS

SEQ ID NO: 3 - protein, 17 aa, Artificial Sequence
  Human Myelin Basic Protein (102-118)
GREDNTFKDRPSESDEL

SEQ ID NO: 4 - protein, 18 aa, Artificial Sequence
  Semliki Forest Virus Epitope (115-129)
IQDTRNAVRACRIQYHHD

SEQ ID NO: 5 - protein, 21 aa, Artificial Sequence
  MOG 35-55
MEVGWYRSPFSRVVHLYRNGK

SEQ ID NO: 6 - protein, 4 aa, Artificial Sequence
  HYG copolymer
HYGH

SEQ ID NO: 7 - protein, 5 aa, Artificial Sequence
  HYG copolymer
HYGHG

SEQ ID NO: 8 - protein, 6 aa, Artificial Sequence
  HYG copolymer
HYGHYG

SEQ ID NO: 9 - protein, 7 aa, Artificial Sequence
  HYG copolymer
HYGHYGH

SEQ ID NO: 10 - protein, 8 aa, Artificial Sequence
  HYG copolymer
HYGHYGHY

SEQ ID NO: 11 - protein, 9 aa, Artificial Sequence
  HYG copolymer
HYGHYGHYG

SEQ ID NO: 12 - protein, 10 aa, Artificial Sequence
  HYG copolymer
HYGHYGHYGH

SEQ ID NO: 13 - protein, 11 aa, Artificial Sequence
  HYG copolymer
HYGHYGHYGHY

SEQ ID NO: 14 - protein, 12 aa, Artificial Sequence
  HYG copolymer
HYGHYGHYGHYG

SEQ ID NO: 15 - protein, 13 aa, Artificial Sequence
  HYG copolymer
HYGHYGHYGHYGH

SEQ ID NO: 16 - protein, 14 aa, Artificial Sequence
  HYG copolymer
HYGHYGHYGHYGHY

SEQ ID NO: 17 - protein, 15 aa, Artificial Sequence
  HYG copolymer
HYGHYGHYGHYGHYG

SEQ ID NO: 18 - protein, 4 aa, Artificial Sequence
  GRE copolymer
GREG

SEQ ID NO: 19 - protein, 5 aa, Artificial Sequence
  GRE copolymer
GREGR

SEQ ID NO: 20 - protein, 6 aa, Artificial Sequence
  GRE copolymer
GREGRE

SEQ ID NO: 21 - protein, 7 aa, Artificial Sequence
  GRE copolymer
GREGREG

SEQ ID NO: 22 - protein, 8 aa, Artificial Sequence
  GRE copolymer
GREGREGR

SEQ ID NO: 23 - protein, 9 aa, Artificial Sequence
  GRE copolymer
GREGREGRE

SEQ ID NO: 24 - protein, 10 aa, Artificial Sequence
  GRE copolymer
GREGREGREG

SEQ ID NO: 25 - protein, 11 aa, Artificial Sequence
  GRE copolymer
GREGREGREGR

SEQ ID NO: 26 - protein, 12 aa, Artificial Sequence
  GRE copolymer
GREGREGREGRE

SEQ ID NO: 27 - protein, 13 aa, Artificial Sequence
  GRE copolymer
GREGREGREGREG

SEQ ID NO: 28 - protein, 14 aa, Artificial Sequence
  GRE copolymer
GREGREGREGREGR

SEQ ID NO: 29 - protein, 15 aa, Artificial Sequence
  GRE copolymer
GREGREGREGREGRE

SEQ ID NO: 30 - protein, 4 aa, Artificial Sequence
  GKH copolymer
GKHG

SEQ ID NO: 31 - protein, 5 aa, Artificial Sequence
  GKH copolymer
GKHGK

SEQ ID NO: 32 - protein, 6 aa, Artificial Sequence
  GKH copolymer
GKHGKH

SEQ ID NO: 33 - protein, 7 aa, Artificial Sequence
  GKH copolymer
GKHGKHG

SEQ ID NO: 34 - protein, 8 aa, Artificial Sequence
  GKH copolymer
GKHGKHGK

SEQ ID NO: 35 - protein, 9 aa, Artificial Sequence
  GKH copolymer
GKHGKHGKH

SEQ ID NO: 36 - protein, 10 aa, Artificial Sequence
  GKH copolymer
GKHGKHGKHG

SEQ ID NO: 37 - protein, 11 aa, Artificial Sequence
  GKH copolymer
GKHGKHGKHGK

SEQ ID NO: 38 - protein, 12 aa, Artificial Sequence
  GKH copolymer
GKHGKHGKHGKH

SEQ ID NO: 39 - protein, 13 aa, Artificial Sequence
  GKH copolymer
GKHGKHGKHGKHG

SEQ ID NO: 40 - protein, 14 aa, Artificial Sequence
  GKH copolymer
GKHGKHGKHGKHGK

SEQ ID NO: 41 - protein, 15 aa, Artificial Sequence
  GKH copolymer
GKHGKHGKHGKHGKH

SEQ ID NO: 42 - protein, 12 aa, Artificial Sequence
  E2 peptide
TRNAVRACRIQY

SEQ ID NO: 43 - protein, 12 aa, Artificial Sequence
  MOG peptide
GDEAELPCRISP

SEQ ID NO: 44 - protein, 13 aa, Artificial Sequence
  E2 peptide
GREKFTIRPHYGK

SEQ ID NO: 45 - protein, 12 aa, Artificial Sequence
  MBP peptide
THYGSLPQKSQR

SEQ ID NO: 46 - protein, 12 aa, Artificial Sequence
  MBP peptide
GSGKDSHTRTTH

SEQ ID NO: 47 - protein, 15 aa, Artificial Sequence
  E2 peptide
IQDTRNAVRASRIQY

SEQ ID NO: 48 - protein, 15 aa, Artificial Sequence
  MOG peptide
DEAELPSRISPGKNA

SEQ ID NO: 49 - protein, 14 aa, Artificial Sequence [sequence omitted]